(19) United States
(12) Patent Application Publication
(10) Pub. No.: US 2024/0366787 A1
(43) Pub. Date: Nov. 7, 2024

(54) Gene therapy DNA vector based on gene therapy DNA vector VTvaf17 carrying the therapeutic gene selected from the group of NOS2, NOS3, VIP, KCNMA1, and CGRP genes for increasing the expression level of these therapeutic genes, method of its production and use, Escherichia coli strain SCS110-AF/VTvaf17-NOS2, or Escherichia coli strain SCS110-AF/VTvaf17-NOS3, or Escherichia coli strain SCS110-AF/VTvaf17-VIP, or Escherichia coli strain SCS110-AF/VTvaf17-KCNMA1, or Escherichia coli strain SCS110-AF/V

(71) Applicants: CELL AND GENE THERAPY LTD, London (GB); OBSCHESTVO S OGRANICHENNOI OTVETSTVENNOSTJU "PRORYVNYE INNOVATSIONNYE TEKHNOLOGII", Moscow (RU)

(72) Inventor: Natalia SAVELIEVA, Wienna (AT)

(21) Appl. No.: 18/268,711

(22) PCT Filed: Dec. 18, 2019
(86) PCT No.: PCT/RU2019/000969

§ 371 Date: Jun. 21, 2023

(30) Foreign Application Priority Data
Dec. 21, 2018 (RU) 2018145694

(51) Int. Cl. A61K 48/00; A61K 38/17; A61K 38/22; A61K 38/26; A61K 38/44; C12N 15/70; C12N 15/85

(52) U.S. Cl. CPC A61K 48/005; A61K 38/1709; A61K 38/225; A61K 38/26; A61K 38/44; C12N 15/70; C12N 15/85; C12Y 114/13039

(57) ABSTRACT

The invention refers to genetic engineering and can be used in biotechnology, medicine, and agriculture for the manufacture of gene therapy products. Gene therapy DNA vector based on the gene therapy DNA vector VTvaflV carrying the therapeutic gene selected from the group of NOS2, NOS3, VIP, KCNMA1, and CGRP genes is constructed in order to increase the expression level of this therapeutic gene in humans and animals, while gene therapy DNA vector VTvafl7-NOS2, or VTvaflV-NOS3, or VTvafl7-VIP, or VTvafl7-KCNMAI, or VTvafl7-CGRP has the nucleotide sequence SEQ ID No. 1, or SEQ ID No. 2, or SEQ ID No. 3, or SEQ ID No. 4, or SEQ ID No. 5, respectively.

### FIELD OF THE INVENTION

The invention refers to genetic engineering and can be used in biotechnology, medicine, and agriculture for the manufacture of gene therapy products.

### BACKGROUND OF THE INVENTION

Gene therapy is an innovative approach in medicine aimed at treating inherited and acquired diseases by means of delivery of new genetic material into a patient's cells to compensate for or suppress the function of a mutant gene and/or treat a genetic disorder. The final product of gene expression may be an RNA molecule or a protein molecule. However, most physiological processes in the body are associated with the functional activity of protein molecules, while RNA molecules are either an intermediate product in the synthesis of proteins or perform regulatory functions. Thus, the objective of gene therapy in most cases is to inject the organism with genes that provide transcription and further translation of protein molecules encoded by these genes. Within the description of the invention, gene expression refers to the production of a protein molecule with amino acid sequence encoded by this gene.

NOS2, NOS3, KCNMA1, VIP, and CGRP genes included in the group of genes play a key role in several processes in human and animal organisms. The correlations between low/insufficient concentrations of these proteins and various human diseases in some cases confirmed by disturbances in normal gene expression encoding these proteins was demonstrated. Thus, the gene therapy upregulation of expression of a gene selected from the group of NOS2, NOS3, KCNMA1, VIP, and CGRP genes has potential to correct various conditions in humans and animals.

The NOS2 gene encodes a protein, namely inducible nitric oxide synthase 2 (synonymous names: NOS2, or NOS2A, or iNOS, or NOS II). The NOS2 protein is involved in the nitric oxide synthesis, which in turn is a mediator of a number of signalling pathways in humans and animals, including ERK and Apelin signalling cascades. The processes in which NOS2 μlays an important role include neurotransmission, antibacterial and antitumour immunity, and vasomotion of different organs and tissues. A connection between the polymorphism and deviations from normal NOS2 gene expression and the susceptibility to different diseases and pathological conditions was found.

For example, it was shown that the relatively low level of NOS2 expression in infants is one of the reasons for the high incidence of STEC hemolytic uremic syndrome (HUS), leading to kidney failure (Tsuji S et al.//Tohoku J Exp Med. 2012 November; 228(3):247-52). Stimulation of nitric oxide synthesis had a protective effect on the Stx-induced HUS mouse model in the course of in vivo experiments (Dran G I et al.//Kidney Int. 2002 October; 62(4):1338-48). In another study the increased likelihood of death from sepsis was observed in mice with reduced NOS2 expression (Cobb J et al.//Surgery 126 (1999) 438-442).

It is known that the reduced NOS2 expression in adenoids was observed in children with otitis media with effusion, and the induction of NOS2 expression is discussed as a promising therapeutic approach (Granath A et al.//Pediatr Allergy Immunol. 2010 December; 21(8):1151-6).

Despite information about a controversial contribution of NOS2 activity in the pathogenesis of cardiovascular diseases, in the study in rabbits with experimental hyperlipidemia and predisposition to atherosclerosis, it was shown that administration of recombinant adenoviral vector expressing the NOS2 gene resulted in the improvement in vasomotor function, and atheroprotective effect of this approach was manifested in a significant reduction in lipid accumulation in the vascular walls and inflammation in the endothelial cells (Jiang B et al.//Hum Gene Ther. 2012 November; 23(11):1166-75). Increasing NOS2 gene expression, including through viral vectors, can reduce or eliminate intimal hyperplasia after angioplasty (Barbato J E, Tzeng E.//Trends Cardiovasc Med. 2004 October;14(7):267-72).

It was shown that a gene therapy approach using a recombinant adenoviral vector expressing saRNA that enhances NOS2 gene expression restored erectile function in rats (Wang T et al.//J Urol. 2013 August; 190(2):790-8).

As for cancer, it was shown that the growth rate and metastasis of tumours was reduced in mice injected with a vector expressing NOS2 (Schwentker A, Billiar T R.//World J Surg. 2002 Jul;26(7):772-8). When NOS2 is inhibited, the effectiveness of BCG therapy for bladder cancer is significantly reduced, and an increase in the NOS2 expression can be considered as a strategy to improve the effectiveness of BCG therapy (Shah G et al.//Urol Oncol. 2014 Jan;32(1):45. el-9).

Also, NOS2 can be used for treatment of obstructive nephropathy (Chevalier R L.//Kidney Int. 2004 October;66(4):1709-10) and skeletal muscle damage (Igamonti E et al.//J Immunol. 2013 Feb. 15; 190(4):1767-77).

The NOS3 gene encodes a protein, namely endothelial nitric oxide synthase 3 (synonymous names: NOS3, or eNOS, or NOS III). NOS3 protein, just like NOS2 protein, is involved in nitric oxide synthesis, but features constitutive expression in vascular endothelial cells and is one of the main factors that ensure vascular tone. The main signalling cascades in which NOS3 is involved include the Act and EGF/EGFR signalling pathways.

It was shown that with the introduction of vectors expressing the NOS3 gene, reduction in high blood pressure and absence of hyperinsulinemia was observed in rats (Zhao C X et al.//J Pharmacol Exp Ther. 2009 February;328(2):610-20). The intratracheal injection of vectors expressing the NOS3, prostacyclin synthase and VEGF genes resulted in a decrease in pulmonary arterial pressure in experimental animals with pulmonary arterial hypertension. Similar positive results were obtained with the introduction of autologous cells transfected with recombinant vectors and expressing NOS3 (Chen et al.//Heart Lung Circ. 2017 May; 26(5):509-518; Wei L et al.//Hypertens Res. 2013 May; 36(5):414-21).

Also, a gene therapy approach using a recombinant adenoviral vector expressing NOS3 restored the erectile function caused by age-related changes in rats (Bivalacqua T et al.//Int J Impot Res. 2000 September;12 Suppl 3: S8-17).

The KCNMA1 gene encodes a protein, i.e. the pore-forming MaxiK subunit of calcium-dependent potassium channels of the cell membrane, that play a fundamental role, primarily in the functioning of smooth muscle and neuronal excitability. Connection of polymorphism of this gene with various diseases, in particular cardiovascular diseases is also shown. For example, variability in the KCNMA1 gene is a risk factor for the development of myocardial infarction and high blood pressure (Tabarki B et al.//Hum Genet. 2016 November; 135(11):1295-1298).

KCNMA1 gene expression is associated with the secretory function of mucous membranes. It was shown that IFN-γ mediated reduction in mucociliary clearance that constitutes the basis of the pathogenesis of COPD, asthma, and probably pulmonary emphysema can be corrected by increasing the expression of the KCNMA1 gene (Manzanares D et al.//Am J Physiol Lung Cell Mol Physiol. 2014 Mar. 1; 306(5): L453-62).

In erectile dysfunction, DNA vector therapy expressing KCNMA1 successfully completed Phase I clinical trials that resulted in obvious improvement in erectile function in patients that persisted for 24 weeks in some cases (Melman, A et al.//Hum Gene Ther. 2006 Dec;17(12):1165-76). Moreover, the injection of autologous cells transfected with a vector expressing KCNMA1 resulted in a significant improvement in erectile function with the experimental model in rats (He Y et al.//Andrologia. 2014 June;46(5):479-86).

In some types of tumour cells, particularly in intestinal cancer cells, the promoter KCNMA1 gene region is hypermethylated, which leads to a decrease in its expression and probably plays a role in the growth and dissemination of tumour. The KCNMA1 gene upregulation by introducing the KCNMA1 transgene into these cells can change the course of the cancer process (Ma G et al.//Mol Cancer. 2017 Feb. 23; 16(1):46).

It is known that the mode of action of the toxin of the West Asian scorpion (Buthus martensi Karsch) is to block the subunits of calcium channels. Probably, the use of KCNMA1 as a competitor with endogenous molecules for binding to the toxin can be used to develop antidotes to this and other toxins with a similar mode of action. (Tao J et al.//Toxins (Basel). 2014 Apr. 22; 6(4):1419-33).

The VIP gene encodes a VIP protein, a vasoactive intestinal peptide that belongs to the glucagon family. It stimulates the myocardial contractile function, causes vasodilation, increases glycogenolysis, lowers blood pressure and relaxes the smooth muscles of the trachea, stomach, and gall bladder. VIP protein acts as an antimicrobial peptide with antibacterial and antifungal activity (Karim I A et al.//J Neuroimmunol. 2008 Aug. 30; 200(1-2):11-6). It also features immune modulating properties manifested in reducing the effect of pro-inflammatory and enhancing the effect of anti-inflammatory mediators, making it a promising molecule for the treatment of rheumatoid arthritis and other autoimmune diseases (Delgado M et al.//Nat Med. 2001 May; 7(5):563-8).

An increase in insulin secretion in response to an increase in glucose level in rats expressing human VIP in beta cells of pancreas was shown (Kato I at al.//Ann N Y Acad Sci. 1996 Dec. 26; 805:232-42). Gene therapy approach using viral vector expressing VIP has proven effective in an experimental model of type II diabetes in mice (Tasyurek H M et al.//Gene Ther. 2018 Jul;25(4):269-283).

In pulmonary arterial hypertension in rats, monotherapy with recombinant VIP protein was more effective than therapy with bosentan (Hamidi S A et al.//Respir Res. 2011 Oct. 26; 12:141).

Regarding cancer diseases, i was shown that VIP expression inhibited the proliferation of renal carcinoma cells (Vacas E et al.//Biochim Biophys Acta. 2012 October;1823(10):1676-85).

It has also been demonstrated that VIP has a positive effect on the healing of mechanical pulmonary epithelium injury (Guan C X et al.//Peptides. 2006 December; 27(12):3107-14).

Invicorp (Plethora Solutions, London, UK) intracavernous injection drug that constitutes a mixture of VIP recombinant protein and phentolamine mesylate is intended for the treatment of erectile dysfunction and effective in 70% of cases of erectile dysfunction that are resistant to injection therapy with other drugs (Wyllie M G//BJU Int. 2010 September;106(5):723-4).

The CGRP gene encodes the CGRP protein, calcitonin gene-related peptide that belongs to a family of proteins that also includes calcitonin, adrenomedullin, and amylin. CGRP functions include vasodilation and acting as an antimicrobial peptide. CGRP is involved in the development of preeclampsia and has a protective effect on the cardiovascular system (Marquez-Rodas I et al.//J Physiol Biochem. 2006 March;62(1):45-56.). In case of vascular damage when CGRP is overexpressed in injury hotspots, the number of apoptotic cells increases and neointimal hyperplasia is prevented. These properties can be used for prevention of restenosis after various surgical procedures on vessels (Wang W, Sun W, Wang X.//Am J Physiol HeartCirc Physiol. 2004 October;287(4):H1582-9).

Also, CGRP plays a role in bone development, metabolism, and remodelling of tissues around implants, and the injection of a viral vector expressing CGRP led to a positive effect on osseointegration of implants in mice (Xiang L et al.//Bone. 2017 January; 94:135-140). Injection of autologous cells transfected with a viral vector expressing the CGRP gene accelerated regeneration in rats with peripheral bone defects (Fang Z et al.//PLoS One. 2013 Aug. 30; 8(8): e72738).

Injection of a plasmid vector expressing the CGRP gene resulted in a decrease in the incidence of diabetes in mice and significantly reduced the level of hyperglycemia in an experimental model of induced autoimmune diabetes (She F et al.//Sheng Li Xue Bao. 2003 Dec. 25; 55(6):625-32). It was shown that the injection of a viral vector expressing the CGRP gene resulted in the restoration of erectile function in rats (Bivalacqua T J et al.//Biol Reprod. 2001 Nov;65(5):1371-7).

Thus, the background of the Invention suggests that mutations in NOS2, NOS3, KCNMA1, VIP, and CGRP genes or insufficient expression of proteins encoded by these genes are associated with the development of a spectrum of diseases, including, but not limited to, cardiovascular diseases, metabolic diseases, erectile dysfunction, autoimmune diseases, hereditary and acquired pathological disorders, oncological diseases, infectious diseases, and other conditions. This is why NOS2, NOS3, KCNMA1, VIP, and CGRP genes are grouped within this patent. Gene constructs that provide for the expression of proteins encoded by NOS2, NOS3, KCNMA1, VIP and CGRP genes included in the group of genes as part of a particular vector for gene therapy can be used to develop drugs for the treatment of various diseases, including, but not limited to, cardiovascular diseases, metabolic diseases, erectile dysfunction, autoimmune diseases, hereditary and acquired pathological disorders, oncological diseases, infectious diseases, and other conditions.

Moreover, these data suggest that insufficient expression of proteins encoded by NOS2, NOS3, KCNMA1, VIP, and CGRP genes included in the group of genes is associated not only with pathological conditions, but also with a predisposition to their development. Also, these data indicate that insufficient expression of these proteins may not appear explicitly in the form of a pathology that can be unambiguously described within the framework of existing clinical practice standards (for example, using the ICD code), but at the same time cause conditions that are unfavourable for humans and animals and associated with deterioration in the quality of life.

Analysis of approaches to increase the expression of therapeutic genes implies the practicability of use of different gene therapy vectors.

Gene therapy vectors are divided into viral, cell, and DNA vectors (Guideline on the quality, non-clinical, and clinical aspects of gene therapy medicinal Products

EMA/CAT/80183/2014). Recently, gene therapy has paid increasingly more attention to the development of non-viral gene delivery systems with plasmid vectors topping the list. Plasmid vectors are free of limitations inherent in cell and viral vectors. In the target cell, they exist as an episome without being integrated into the genome, while producing them is quite cheap, and there is no immune response or side effects caused by the administration of plasmid vectors, which makes them a convenient tool for gene therapy and prevention of the genetic diseases (DNA vaccination) (Li L, Petrovsky N.//Expert Rev Vaccines. 2016; 15(3):313-29).

However, limitations of plasmid vectors use in gene therapy are: 1) presence of antibiotic resistance genes for the production of constructs in bacterial strains; 2) the presence of various regulatory elements represented by sequences of viral genomes; 3) length of therapeutic plasmid vector that determines the efficiency of vector delivery to the target cell.

It is known that the European Medicines Agency deems it necessary to refrain from adding antibiotic resistance marker genes to newly engineered plasmid vectors for gene therapy (Reflection paper on design modifications of gene therapy medicinal products during development/14 Dec. 2011 EMA/CAT/GTWP/44236/2009 Committee for advanced therapies). This recommendation is primarily related to the potential danger of the DNA vector penetration or horizontal antibiotic resistance gene transfer into the cells of bacteria found in the body as part of normal or opportunistic microflora. Furthermore, the presence of antibiotic resistance genes significantly increases the length of DNA vector, which reduces the efficiency of its penetration into eukaryotic cells.

It is important to note that antibiotic resistance genes also make a fundamental contribution to the method of production of DNA vectors. If antibiotic resistance genes are present, strains for the production of DNA vectors are usually cultured in medium containing a selective antibiotic, which poses risk of antibiotic traces in insufficiently purified DNA vector preparations. Thus, production of DNA vectors for gene therapy without antibiotic resistance genes is associated with the production of strains with such distinctive feature as the ability for stable amplification of therapeutic DNA vectors in the antibiotic-free medium.

In addition, the European Medicines Agency recommends avoiding the presence of regulatory elements in therapeutic plasmid vectors to increase the expression of therapeutic genes (promoters, enhancers, post-translational regulatory elements) that constitute nucleotide sequences of genomes of various viruses (Draft Guideline on the quality, non-clinical and clinical aspects of gene therapy medicinal products,http://www.ema.europa.eu/docs/en_GB/document_library/Scientific_guideline/2015/05/WC500187020.pdf). Although these sequences can increase the expression level of the therapeutic transgene, however, they pose risk of recombination with the genetic material of wild-type viruses and integration into the eukaryotic genome. Moreover, the relevance of overexpression of the particular gene for therapy remains an unresolved issue.

The size of the therapy vector is also essential. It is known that modern plasmid vectors often have unnecessary, non-functional sites that increase their length substantially (Mairhofer J, Grabherr R.//Mol Biotechnol. 2008.39(2):97-104). For example, ampicillin resistance gene in pBR322 series vectors, as a rule, consists of at least 1000 bp, which is more than 20% of the length of the vector itself. A reverse relationship between the vector length and its ability to penetrate into eukaryotic cells is observed; DNA vectors with a small length effectively penetrate into human and animal cells. For example, in a series of experiments on transfection of HELA cells with 383-4548 bp DNA vectors it was shown that the difference in penetration efficiency can be up to two orders of magnitude (100 times different) (Hornstein B D et al.//PLoS ONE. 2016; 11(12): e0167537.).

Thus, when selecting a DNA vector, for reasons of safety and maximum effectiveness, preference should be given to those constructs that do not contain antibiotic resistance genes, the sequences of viral origin and length of which allows for the effective penetration into eukaryotic cells. A strain for production of such DNA vector in quantities sufficient for the purposes of gene therapy should ensure the possibility of stable DNA vector amplification using antibiotic-free nutrient media.

Example of usage of the recombinant DNA vectors for gene therapy is the method of producing a recombinant vector for genetic immunisation (Patent No. U.S. Pat. No. 9,550,998 B2). The plasmid vector is a supercoiled plasmid DNA vector that is used for the expression of cloned genes in human and animal cells. The vector contains an origin of replication, regulatory elements comprising human cytomegalovirus promoter and enhancer, and regulatory sequences from the human T-cell lymphotropic virus.

The vector is accumulated in a dedicated E. coli strain free of antibiotics through antisense complementation of sacB gene inserted into the strain by means of bacteriophage. The disadvantage of this invention is the presence of regulatory elements in the composition of DNA vector that constitute sequences of viral genomes.

The following examples are prototypes of this invention regarding use of NOS2, NOS3, VIP, KCNMA1, and CGRP genes in order to increase the expression level of these therapeutic genes by the gene therapy method.

Patent No. U.S. Pat. No. 5,594,032A describes a method of treating erectile dysfunction by injecting the cDNA of NOS2 gene that enhances the expression of NOS2 into the organism. The present invention also describes a method for delivering cDNA of NOS2 gene into the body using genetically modified cells injected with cDNA of NOS2 gene. The disadvantage of this invention is the limited use of the invention in the therapy of erectile dysfunction and the lack of a gene therapy approach using different vectors allowing expression of cDNA of NOS2 gene.

Application No. US20040120930A1 describes a technique in the treatment of acute limb ischemia by injecting the recombinant NOS3 protein or vector expressing NOS3 gene into the body. The disadvantages of this invention include the limited use of the invention in the treatment of acute limb ischemia and absence of specific requirements for vectors allowing expression of cDNA of NOS2 gene.

Patent No. U.S. Pat. No. 8,536,146B2 describes a method for modulating nerve cell function by altering the expression of KCNMA1 gene. This method involves reducing the expression of KCNMA1 gene by introducing siRNA into cells. The disadvantages of this invention include the limited further implementation of invention to the states associated with pathologically high KCNMA1 expression, a method for regulating gene expression by using siRNA, and the absence of a gene therapy approach using various vectors that regulate KCNMA1 gene expression.

Application No. WO1994016718A1 describes genetically modified neuronal stem cells for the treatment of central nervous system diseases. Genetically modified neural stem cells may contain a recombinant construct allowing expression of a gene selected from the group of genes, including VIP and CGRP genes. The disadvantages of this invention include the limited further implementation of invention in the treatment of nervous disorders, as well as the use of genetically modified cells to increase VIP and CGRP expression, but not DNA vectors expressing these genes.

### DISCLOSURE OF THE INVENTION

The purpose of this invention is to construct the gene therapy DNA vectors in order to increase the expression level of a group of NOS2, NOS3, VIP, KCNMA1, and CGRP genes in human and animal organisms that combine the following properties:
I) Efficiency of gene therapy DNA vector in order to increase the expression level of therapeutic genes in eukaryotic cells.
II) Possibility of safe use in gene therapy of human beings and animals due to the absence of regulatory elements representing the nucleotide sequences of viral genomes in the gene therapy DNA vector.
III) Possibility of safe use in the gene therapy of human beings and animals due to the absence of antibiotic resistance genes in the gene therapy DNA vector.
IV) Producibility and constructability of gene therapy DNA vector on an industrial scale.

Item II and III are provided for herein in line with the recommendations of the state regulators for gene therapy medicines and, specifically, the requirement of the European Medicines Agency to refrain from adding antibiotic resistance marker genes to newly engineered plasmid vectors for gene therapy (Reflection paper on design modifications of gene therapy medicinal products during development/14 Dec. 2011 EMA/CAT/GTWP/44236/2009 Committee for advanced therapies) and refrain from adding viral genomes to newly engineered plasmid vectors for gene therapy (Guideline on the quality, non-clinical and clinical aspects of gene therapy medicinal products/23 Mar. 2015, EMA/CAT/80183/2014, Committee for Advanced Therapies).

The purpose of the invention also includes the construction of strains carrying these gene therapy DNA vectors for the development and production of these gene therapy DNA vectors on an industrial scale.

The specified purpose is achieved by using the produced gene therapy DNA vector based on the gene therapy DNA vector VTvafl7 for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction, while the gene therapy DNA vector VTvafl7-NOS2 contains the coding region of NOS2 therapeutic gene cloned to gene therapy DNA vector VTvafl7 with the nucleotide sequence SEQ ID No. 1, the gene therapy DNA vector VTvafl7-NOS3 contains the coding region of NOS3 therapeutic gene cloned to gene therapy DNA vector VTvafl7 with the nucleotide sequence SEQ ID No. 2, the gene therapy DNA vector VTvafl7-VIP contains the coding region of VIP therapeutic gene cloned to gene therapy DNA vector VTvafl7 with the nucleotide sequence SEQ ID No. 3, the gene therapy DNA vector VTvafl7-KCNMA1 contains the coding region of KCNMA1 therapeutic gene cloned to gene therapy DNA vector VTvafl7 with the nucleotide sequence SEQ ID No. 4, the gene therapy DNA vector VTvafl7-CGRP contains the coding region of CGRP therapeutic gene cloned to gene therapy DNA vector VTvafl7 with the nucleotide sequence SEQ ID No. 5.

Each of the constructed gene therapy DNA vectors, namely VTvafl7-NOS2, or VTvafl7-NOS3, or VTvafl7-VIP, or VTvafl7-KCNMA1, or VTvafl7-CGRP due to the limited size of VTvafl7 vector part not exceeding 3200 bp has the ability to efficiently penetrate into human and animal cells and express the NOS2, or NOS3, or VIP, or KCNMA1, or CGRP therapeutic gene cloned to it.

Each of the constructed gene therapy DNA vectors, namely VTvafl7-NOS2, or VTvafl7-NOS3, or VTvafl7-VIP, or VTvafl7-KCNMA1, or VTvafl7-CGRP uses nucleotide sequences that are not antibiotic resistance genes, virus genes, or regulatory elements of viral genomes as the structure elements, which ensures its safe use for gene therapy in humans and animals.

A method of gene therapy DNA vector production based on gene therapy DNA vector VTvafl7 carrying the NOS2, NOS3, VIP, KCNMA1, CGRP therapeutic gene was also developed that involves obtaining each of gene therapy DNA vectors: VTvafl7-NOS2, or VTvafl7-NOS3, or VTvafl7-VIP, or VTvafl7-KCNMA1, or VTvafl7-CGRP as follows: the coding region of the NOS2, NOS3, VIP, KCNMA1, CGRP therapeutic gene is cloned to gene therapy DNA vector VTvafl7, and gene therapy DNA vector VTvafl7-NOS2, SEQ ID No. 1, or VTvafl7-NOS3, SEQ ID No. 2, or VTvafl7-VIP, SEQ ID No. 3, or VTvafl7-KCNMA1, SEQ ID No. 4 or VTvafl7-CGRP, SEQ ID No. 5, respectively, is obtained, while the coding region of the NOS2, or NOS3, or VIP, or KCNMA1, or CGRP therapeutic gene is obtained by isolating total RNA from the human biological tissue sample followed by the reverse transcription reaction and PCR amplification using the obtained oligonucleotides and cleaving the amplification product by corresponding restriction endonucleases, while cloning to the gene therapy DNA vector VTvafl7 is performed by SalI and KpnI, or BamHI and EcoRI restriction sites, while the selection is performed without antibiotics,
at the same time, the following oligonucleotides produced for this purpose are used during gene therapy DNA vector VTvafl7-NOS2, SEQ ID No. 1 production for the reverse transcription reaction and PCR amplification:

NOS2_F ATCGTCGACCACCATGGCCTGTCCTTGGAAATTTC,
NOS2_R CGGTACCTCAGAGCGCTGACATCTCCAGG,

and the cleaving of amplification product and cloning of the coding region of NOS2 gene to gene therapy DNA vector VTvafl7 is performed by SalI and KpnI restriction endonucleases,
at the same time, the following oligonucleotides produced for this purpose are used during gene therapy DNA vector VTvafl7-NOS3, SEQ ID No. 2 production for the reverse transcription reaction and PCR amplification:

NOS3_F GACAAGCTTCCACCATGGGCAACTTGAAGAG,
NOS3_R GGAATTCAGGGGCTGTTGGTGTCTGAGCCG,

and the cleaving of amplification product and cloning of the coding region of NOS3 gene to gene therapy DNA vector VTvafl7 is performed by HindIIII [symbol] EcoRI restriction endonucleases,
at the same time, the following oligonucleotides produced for this purpose are used during gene therapy DNA vector VTvafl7-VIP, SEQ ID No. 3 production for the reverse transcription reaction and PCR amplification:

VIP_F AGGATCCACCATGGACACCAGAAATAAGGCCCAG,
VIP_R GGAATTCATTTTTCTAACTCTTCTGGAAAG,

and the cleaving of amplification product and cloning of the coding region of VIP gene to gene therapy DNA vector VTvafl7 is performed by BamHI [symbol] EcoRI restriction endonucleases,
at the same time, the following oligonucleotides produced for this purpose are used during gene therapy DNA vector VTvafl7-KCNMA1, SEQ ID No. 4 production for the reverse transcription reaction and PCR amplification:

KCNMA1_F
AGGATCCGGTACCGAGGAGATCTGCCGCCGCGATCGCCATG,
KCNMA1_R ACCAAGCTTATCTGTAAACCATTTCTTTTCTG,

and the cleaving of amplification product and cloning of the coding region of KCNMA1 gene to gene therapy DNA vector VTvafl7 is performed by BamHI [symbol] EcoRI restriction endonucleases,
at the same time, the following oligonucleotides produced for this purpose are used during gene therapy DNA vector VTvafl7-CGRP, SEQ ID No. 5 production for the reverse transcription reaction and PCR amplification:

CGRP_F
AGGATCCGGACGTCATGGAAGTGAAGGATGCCAATT,
CGRP_R
GGAATTCCTATGCTGGGTCCTCTTCGTCCATTG,

and the cleaving of amplification product and cloning of the coding region of CGRP gene to gene therapy DNA vector VTvafl7 is performed by BamHI [symbol] EcoRI restriction endonucleases.

A method of use of the gene therapy DNA vector based on gene therapy DNA vector VTvafl7 carrying NOS2, NOS3, VIP, KCNMA1, and CGRP therapeutic gene for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction was developed that involves transfection of the cells of patient or animal organs and tissues with the selected gene therapy DNA vector carrying the therapeutic gene based on gene therapy DNA vector VTvafl7, or several selected gene therapy DNA vectors carrying therapeutic genes based on gene therapy DNA vector VTvafl7, from the group of constructed gene therapy DNA vectors carrying therapeutic genes based on gene therapy DNA vector VTvafl7 and/or injection of autologous cells of said patient or animal transfected by the selected gene therapy DNA vector carrying therapeutic gene based on gene therapy DNA vector VTvafl7 or several selected gene therapy DNA vectors carrying the therapeutic genes based on gene therapy DNA vector VTvafl7 from the constructed gene therapy DNA vectors carrying therapeutic genes based on gene therapy DNA vector VTvafl7 into the organs and tissues of the same patient or animal and/or the injection of the selected gene therapy DNA vector carrying therapeutic gene based on gene therapy DNA vector VTvafl7 or several selected gene therapy DNA vectors carrying therapeutic genes based on gene therapy DNA vector VTvafl7 from the group of constructed gene therapy DNA vectors carrying therapeutic genes based on gene therapy DNA vector VTvafl7 into the organs and tissues of the same patient or animal, or the combination of the indicated methods.

A method of production of strain for construction of a gene therapy DNA vector for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction was developed that involves making electrocompetent cells of Escherichia coli strain SCS110-AF and subjecting these cells to electroporation with gene therapy DNA vector VTvafl7-NOS2, or gene therapy DNA vector VTvafl7-NOS3, or gene therapy DNA vector VTvafl7-VIP, or gene therapy DNA vector VTvafl7-KCNMA1, or gene therapy DNA vector VTvafl7-CGRP. After that, the cells are poured into agar plates (Petri dishes) with a selective medium containing yeastrel, peptone, 6% sucrose, and 10 μg/ml of chloramphenicol, and as a result, Escherichia coli strain SCS110-AF/VTvafl7-NOS2 or Escherichia coli strain SCS110-AF/VTvafl7-NOS3, or Escherichia coli strain SCS110-AF/VTvafl7-VIP, or Escherichia coli strain SCS110-AF/VTvafl7-KCNMA1, or Escherichia coli strain SCS110-AF/VTvafl7-CGRP is obtained.

Escherichia coli strain SCS110-AF/VTvafl7-NOS2 carrying the gene therapy DNA vector VTvafl7-NOS2 for production thereof allowing for antibiotic-free selection during gene therapy DNA vector production, or Escherichia coli strain SCS110-AF/VTvafl7-NOS3 carrying the gene therapy DNA vector VTvafl7-NOS3 for production thereof allowing for antibiotic-free selection during gene therapy DNA vector production, or Escherichia coli strain SCS110-AF/VTvafl7-VIP carrying the gene therapy DNA vector VTvafl7-VIP for production thereof allowing for antibiotic-free selection during gene therapy DNA vector production, or Escherichia coli strain SCS110-AF/VTvafl7-KCNMA1 carrying the gene therapy DNA vector VTvafl7-KCNMA1 for production thereof allowing for antibiotic-free selection during gene therapy DNA vector production, or Escherichia coli strain SCS110-AF/VTvafl7-CGRP carrying the gene therapy DNA vector VTvafl7-CGRP for production thereof allowing for antibiotic-free selection during gene therapy DNA vector production is claimed for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction.

A method of production on an industrial scale of gene therapy DNA vector based on gene therapy DNA vector VTvafl7 carrying the NOS2, or NOS3, or VIP, or KCNMA1, or CGRP therapeutic gene for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction was developed that involves production of gene therapy DNA vector VTvafl7-NOS2, or gene therapy DNA vector VTvafl7-NOS3, or gene therapy DNA vector VTvafl7-VIP, or gene therapy DNA vector VTvafl7-KCNMA1, or gene therapy DNA vector VTvafl7-CGRP by inoculating a culture flask containing the prepared medium with seed culture selected from Escherichia coli strain CS110-AF/VTvafl7-NOS2, or Escherichia coli strain SCS110-AF/VTvafl7-NOS3, or Escherichia coli strain SCS110-AF/VTvafl7-VIP, or Escherichia coli strain SCS110-AF/VTvafl7-KCNMAI, or Escherichia coli strain SCS110-AF/VTvafl7-CGRP then the cell culture is incubated in an incubator shaker and transferred to an industrial fermenter, then grown to a stationary phase, then the fraction containing the target DNA product is extracted, multi-stage filtered, and purified by chromatographic methods.

### BRIEF DESCRIPTION OF THE DRAWINGS

The essence of the invention is explained in the drawings, where:

FIG. 1 shows the structure of gene therapy DNA vector VTvafl7 carrying the therapeutic gene selected from the group of NOS2, NOS3, VIP, KCNMA1, and CGRP genes that constitutes a circular double-stranded DNA molecule capable of autonomous replication in Escherichia coli cells.

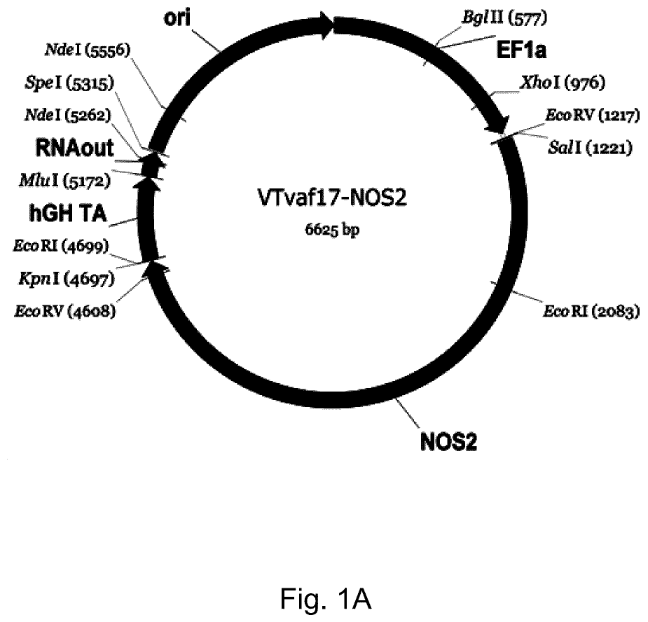

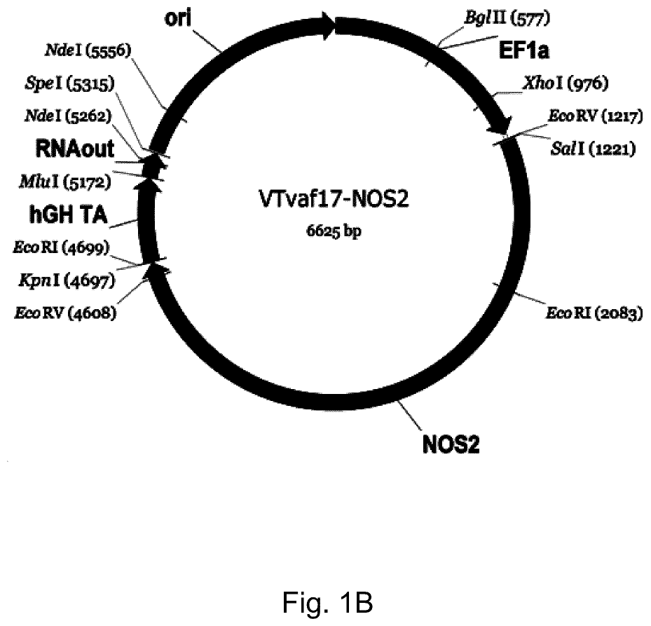

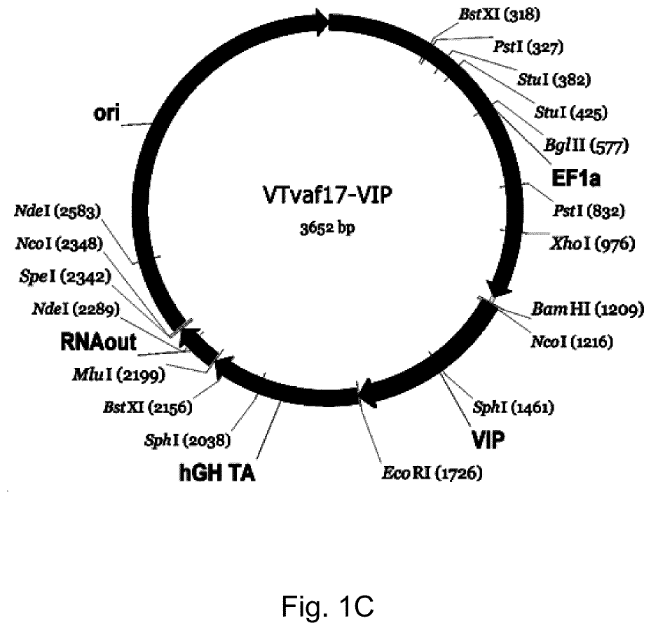

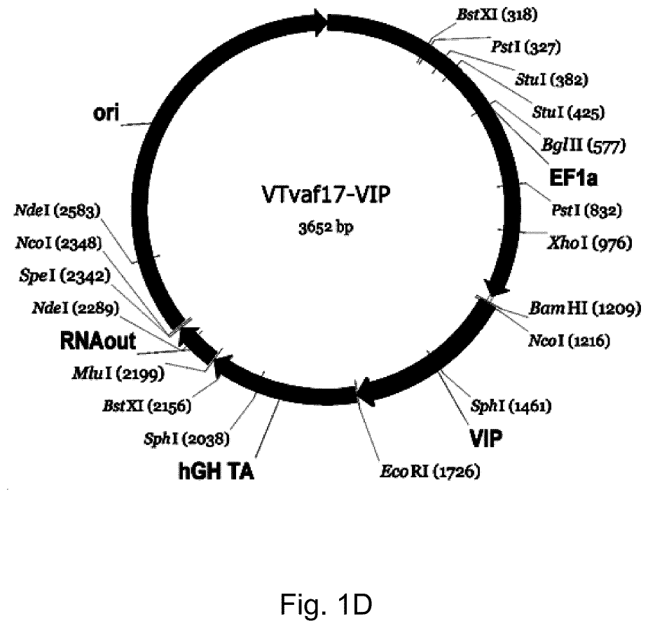

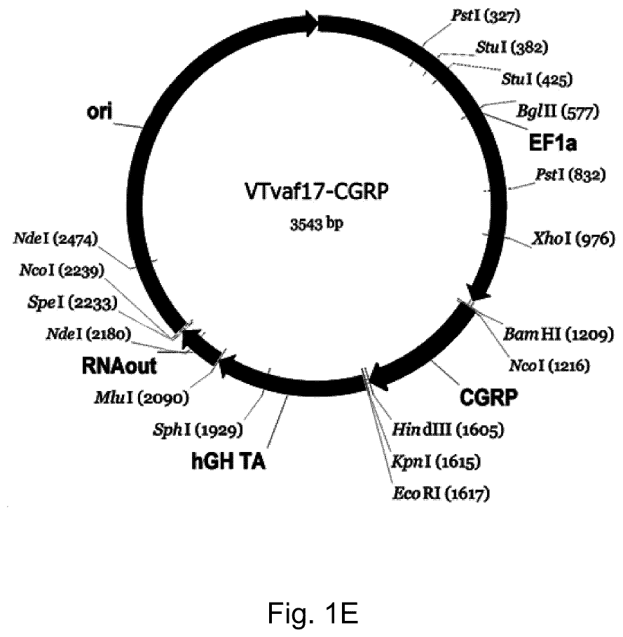

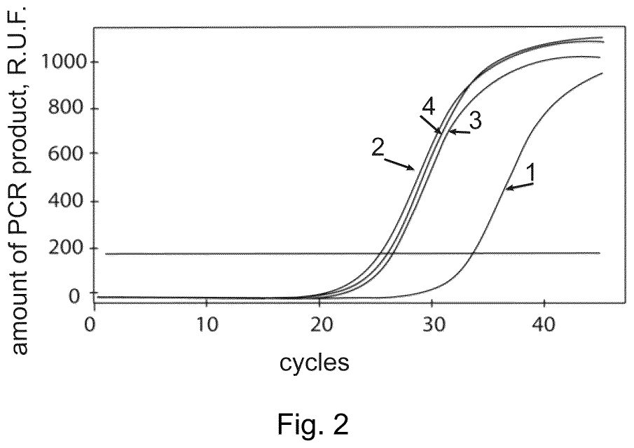

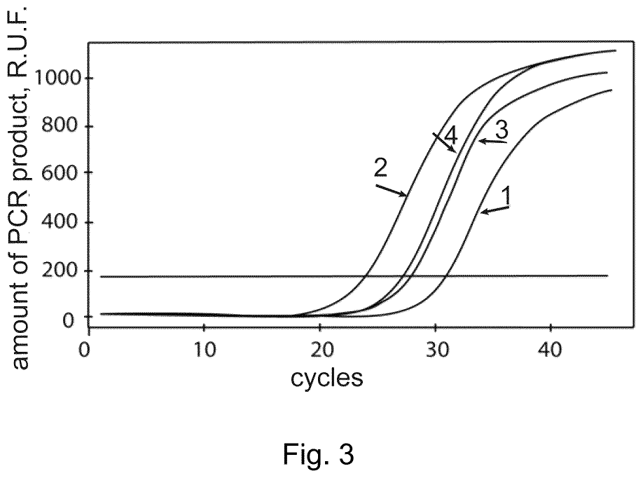

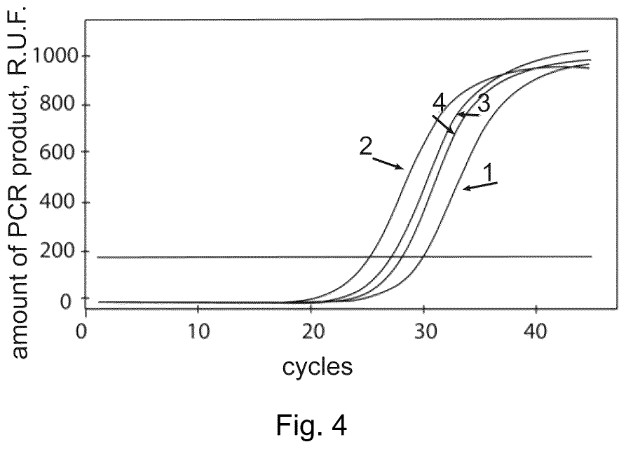

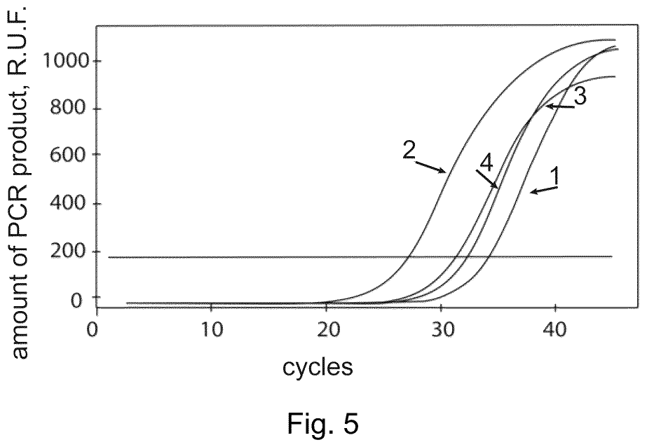

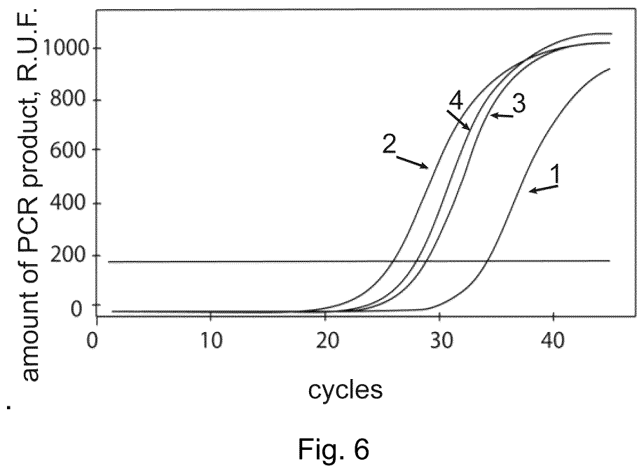

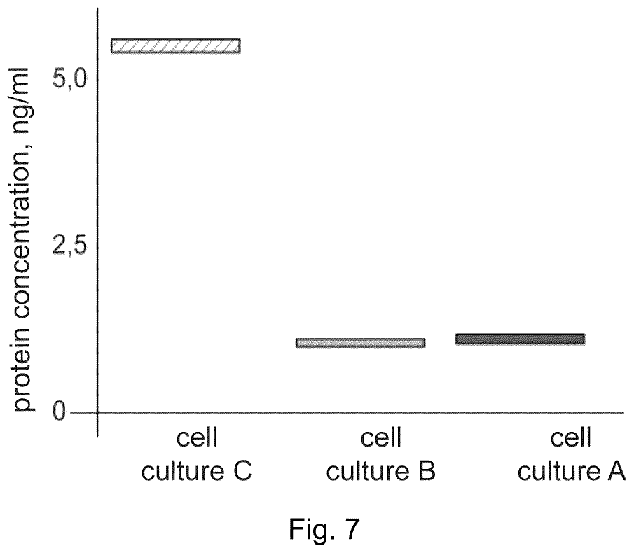

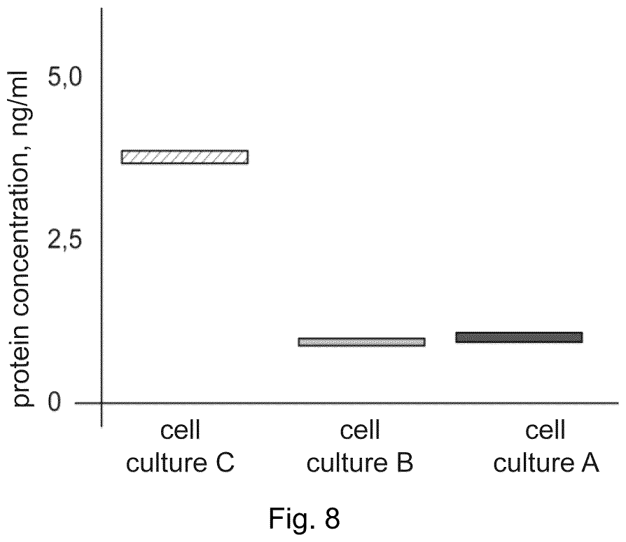

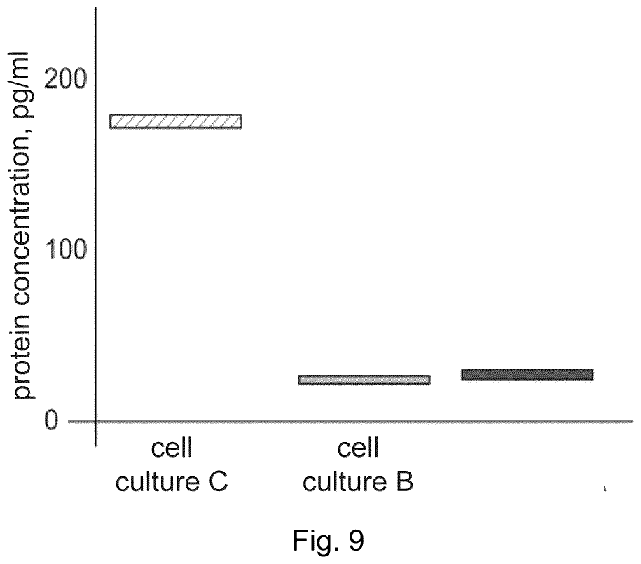

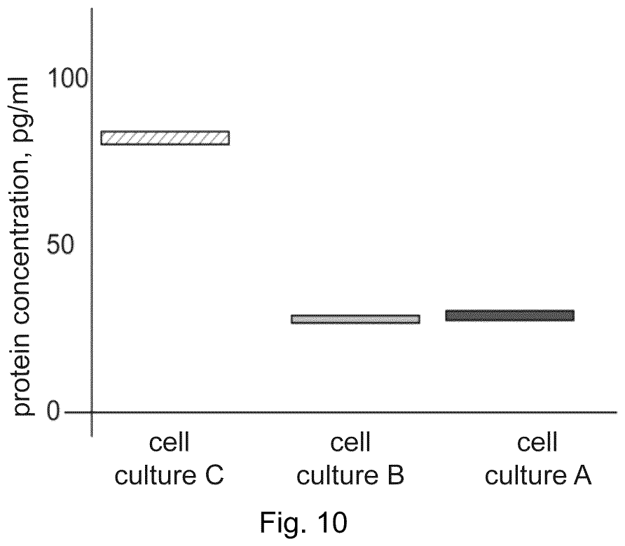

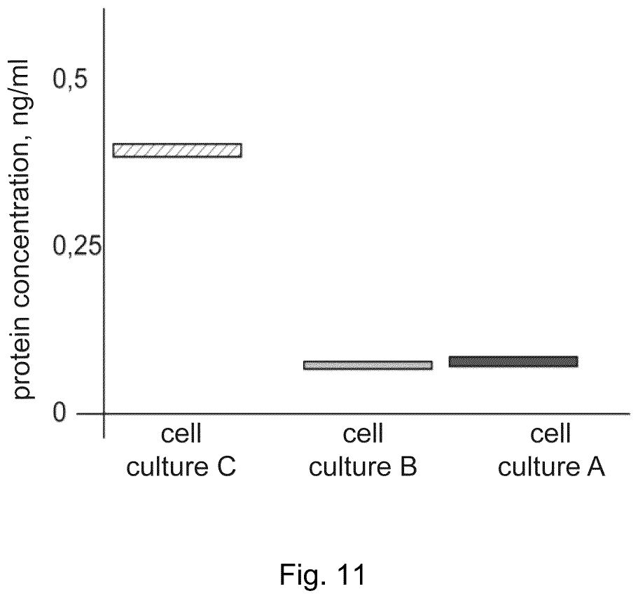

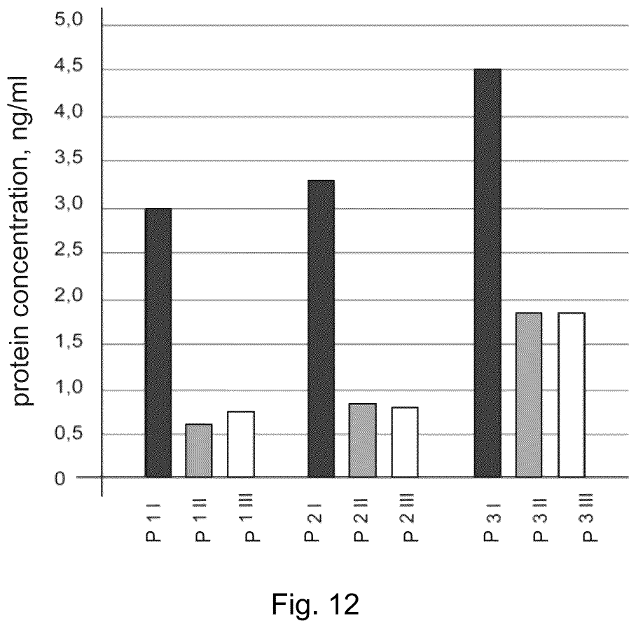

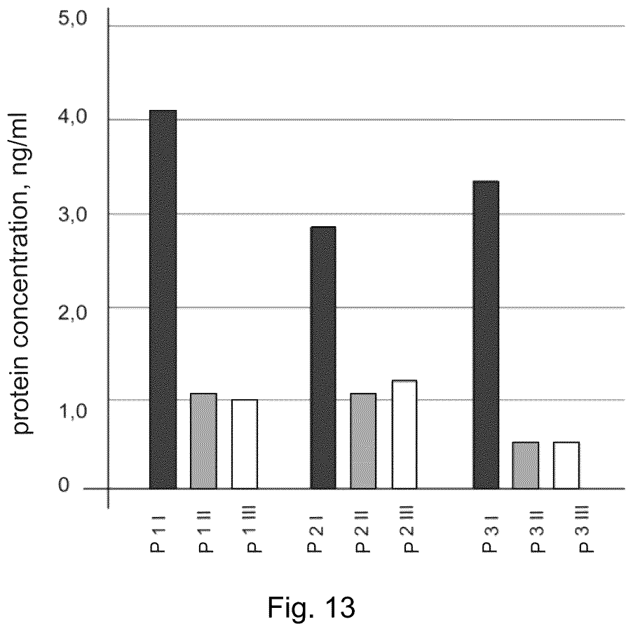

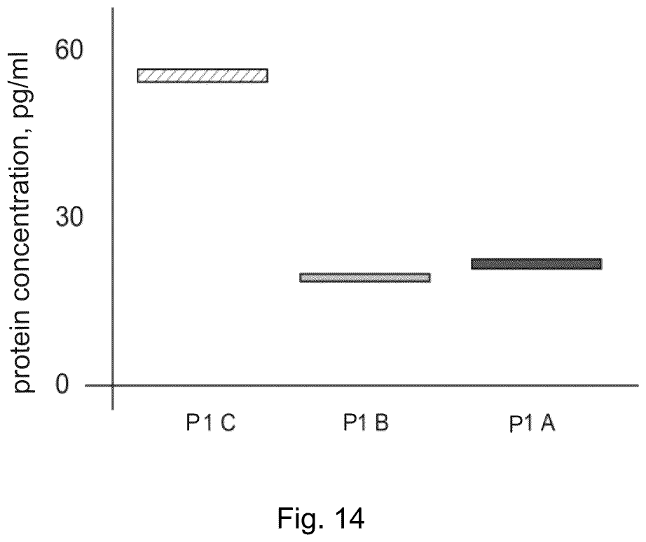

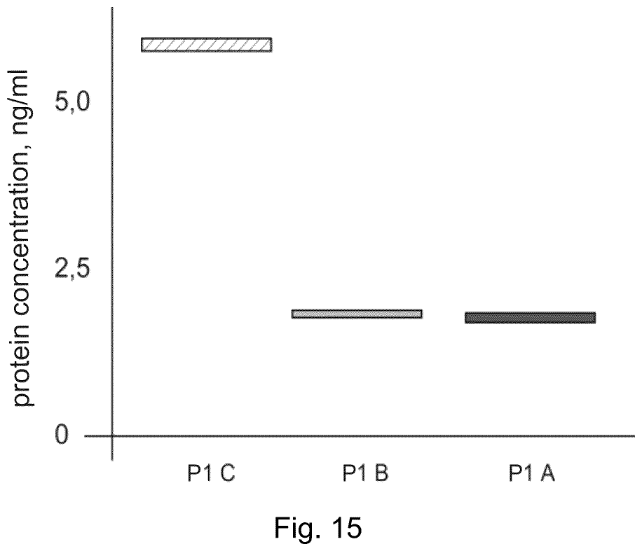

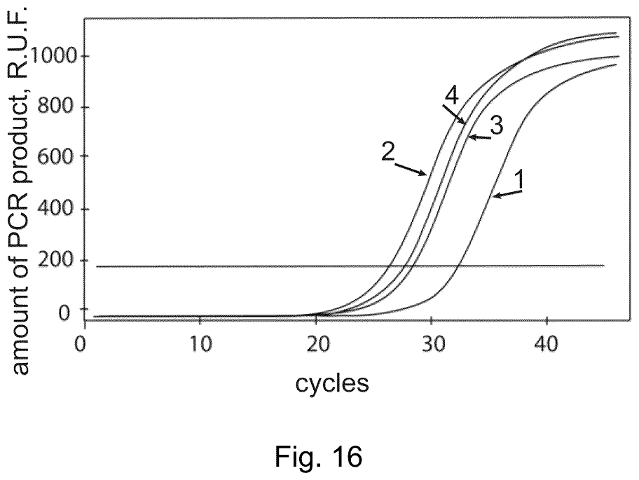

FIG. 1 shows the structures corresponding to:
A—gene therapy DNA vector VTvafl7-NOS2,
B—gene therapy DNA vector VTvafl7-NOS3,
C—gene therapy DNA vector VTvafl7-VIP,
D—gene therapy DNA vector VTvafl7-KCNMA1,
E—gene therapy DNA vector VTvafl7-CGRP.

The following structural elements of the vector are indicated in the structures: EF1a—the promoter region of human elongation factor EF1A with an intrinsic enhancer contained in the first intron of the gene. It ensures efficient transcription of the recombinant gene in most human tissues,

The reading frame of the therapeutic gene corresponding to the coding region of the NOS2 gene (FIG. 1A), or NOS3 (FIG. 1B), or VIP (FIG. 1C), or KCNMA1 (FIG. 1D), or CGRP (FIG. 1E), respectively,
hGH-TA—the transcription terminator and the polyadenylation site of the human growth factor gene,
ori—the origin of replication for autonomous replication with a single nucleotide substitution to increase plasmid production in the cells of most Escherichia coli strains,
RNA-out—the regulatory element RNA-out of transposon Tn 10 allowing for antibiotic-free positive selection in case of the use of Escherichia coli strain SCS 110-AF.

Unique restriction sites are marked.

FIG. 2 shows diagrams of cDNA amplicon accumulation of the therapeutic gene, namely the NOS2 gene, in HBdSMC human urinary bladder smooth muscle cells (ATCC PCS-420-012) before their transfection and 48 hours after transfection of these cells with gene therapy DNA vector VTvafl7-NOS2 in order to assess the ability to penetrate into eukaryotic cells and functional activity, i.e. expression of the therapeutic gene at the mRNA level.

The following curves of accumulation of amplicons during the reaction are shown in FIG. 2 corresponding to:
1—cDNA of NOS2 gene in HBdSMC primary human urinary bladder smooth muscle cells before transfection with DNA vector VTvafl7-NOS2,
2—cDNA of NOS2 gene in HBdSMC primary human urinary bladder smooth muscle cells after transfection with DNA vector VTvafl7-NOS2,
3—cDNA of B2M gene in HBdSMC primary human urinary bladder smooth muscle cells before transfection with DNA vector VTvafl7-NOS2,
4—cDNA of B2M gene in HBdSMC primary human urinary bladder smooth muscle cells after transfection with DNA vector VTvafl7-NOS2.

B2M (beta-2-microglobuline) gene listed in the GenBank database under number NM 004048.2 was used as a reference gene.

FIG. 3 shows diagrams of cDNA amplicon accumulation of the therapeutic gene, namely the NOS3 gene, in T/G HA-VSMC primary human aortic smooth muscle cells (ATCC CRL-1999™) before their transfection and 48 hours after transfection of these cells with gene therapy DNA vector VTvafl7-NOS3 in order to assess the ability to penetrate into eukaryotic cells and functional activity, i.e. expression of the therapeutic gene at the mRNA level.

The following curves of accumulation of amplicons during the reaction are shown in FIG. 3 corresponding to:
1—cDNA of NOS3 gene in T/G HA-VSMC primary human aortic smooth muscle cells before transfection with DNA vector VTvafl7-NOS3,
2—cDNA of NOS3 gene in T/G HA-VSMC primary human aortic smooth muscle cells after transfection with DNA vector VTvafl7-NOS3,
3—cDNA of B2M gene in T/G HA-VSMC primary human aortic smooth muscle cells before transfection with DNA vector VTvafl7-NOS3,
4—cDNA of B2M gene in T/G HA-VSMC primary human aortic smooth muscle cells after transfection with DNA vector VTvafl7-NOS3.

B2M (beta-2-microglobuline) gene listed in the GenBank database under number NM 004048.2 was used as a reference gene.

FIG. 4 shows diagrams of cDNA amplicon accumulation of the therapeutic gene, namely the VIP gene, in HBdSMC human urinary bladder smooth muscle cells (ATCC PCS-420-012) before their transfection and 48 hours after transfection of these cells with gene therapy DNA vector VTvafl7-VIP in order to assess the ability to penetrate into eukaryotic cells and functional activity, i.e. expression of the therapeutic gene at the mRNA level.

The following curves of accumulation of amplicons during the reaction are shown in FIG. 4 corresponding to:
1—cDNA of VIP gene in HBdSMC primary human urinary bladder smooth muscle cells before transfection with DNA vector VTvafl7-VIP,
2—cDNA of VIP gene in HBdSMC primary human urinary bladder smooth muscle cells after transfection with DNA vector VTvafl7-VIP,
3—cDNA of B2M gene in HBdSMC primary human urinary bladder smooth muscle cells before transfection with DNA vector VTvafl7-VIP,
4—cDNA of B2M gene in HBdSMC primary human urinary bladder smooth muscle cells after transfection with DNA vector VTvafl7-VIP.

B2M (beta-2-microglobuline) gene listed in the GenBank database under number NM 004048.2 was used as a reference gene.

FIG. 5 shows diagrams of cDNA amplicon accumulation of the therapeutic gene, namely the KCNMAI gene, in primary culture of corpus cavernosum penis smooth muscle cells before their transfection and 48 hours after transfection of these cells with gene therapy DNA vector VTvafl7-KCNMA1 in order to assess the ability to penetrate into eukaryotic cells and functional activity, i.e. expression of the therapeutic gene at the mRNA level.

The following curves of accumulation of amplicons during the reaction are shown in FIG. 5 corresponding to:
1—cDNA of KCNMA1 gene in primary culture of corpus cavernosum penis smooth muscle cells before transfection with DNA vector VTvafl7-KCNMA1,
2—cDNA of KCNMA1 gene in primary culture of corpus cavernosum penis smooth muscle cells after transfection with DNA vector VTvafl7-KCNMA1,
3—cDNA of B2M gene in primary culture of corpus cavernosum penis smooth muscle cells before transfection with DNA vector VTvafl7-KCNMA1,
4—cDNA of B2M gene in primary culture of corpus cavernosum penis smooth muscle cells after transfection with DNA vector VTvafl7-KCNMA1.

B2M (beta-2-microglobuline) gene listed in the GenBank database under number NM 004048.2 was used as a reference gene.

FIG. 6 shows diagrams of cDNA amplicon accumulation of the therapeutic gene, namely the CGRP gene, in HBdSMC human urinary bladder smooth muscle cells (ATCC PCS-420-012) before their transfection and 48 hours after transfection of these cells with gene therapy DNA vector VTvafl7-CGRP in order to assess the ability to penetrate into eukaryotic cells and functional activity, i.e. expression of the therapeutic gene at the mRNA level.

The following curves of accumulation of amplicons during the reaction are shown in FIG. 6 corresponding to:
1—cDNA of CGRP gene in HBdSMC primary human urinary bladder smooth muscle cells before transfection with DNA vector VTvafl7-CGRP,
2—cDNA of CGRP gene in HBdSMC primary human urinary bladder smooth muscle cells after transfection with DNA vector VTvafl7-CGRP,
3—cDNA of B2M gene in HBdSMC primary human urinary bladder smooth muscle cells before transfection with DNA vector VTvafl7-CGRP,
4—cDNA of B2M gene in HBdSMC primary human urinary bladder smooth muscle cells after transfection with DNA vector VTvafl7-CGRP.

B2M (beta-2-microglobuline) gene listed in the GenBank database under number NM_004048.2 was used as a reference gene.

FIG. 7 shows the plot of NOS2 protein concentration in the cell lysate of HBdSMC human urinary bladder smooth muscle cells (ATCC PCS-420-012) after transfection of these cells with DNA vector VTvafl7-NOS2 in order to assess the functional activity, i.e. expression at the protein level based on the NOS2 protein concentration change in the cell lysate.

The following elements are indicated in FIG. 7:
culture A—HBdSMC human urinary bladder smooth muscle cells transfected with aqueous dendrimer solution without DNA vector (reference),
culture B—HBdSMC human urinary bladder smooth muscle cells transfected with DNA vector VTvafl7,
culture C—HBdSMC human urinary bladder smooth muscle cells transfected with DNA vector VTvafl7-NOS2.

FIG. 8 shows the plot of NOS3 protein concentration in the lysate of HBdSMC human urinary bladder smooth muscle cells (ATCC PCS-420-012) after transfection of these cells with gene therapy DNA vector VTvafl7-NOS3 in order to assess the functional activity, i.e. the therapeutic gene expression at the protein level, and the possibility of increasing the level of protein expression by gene therapy DNA vector based on gene therapy DNA vector VTvafl7 carrying the NOS3 therapeutic gene.

The following elements are indicated in FIG. 8:
culture A—HBdSMC human urinary bladder smooth muscle cells transfected with aqueous dendrimer solution without DNA vector (reference),
culture B—HBdSMC human urinary bladder smooth muscle cells transfected with DNA vector VTvafl7,
culture C—HBdSMC human urinary bladder smooth muscle cells transfected with DNA vector VTvafl7-NOS3.

FIG. 9 shows the plot of VIP protein concentration in conditioned medium from HBdSMC human urinary bladder smooth muscle cells (ATCC PCS-420-012) after transfection of these cells with gene therapy DNA vector VTvafl7-VIP in order to assess the functional activity, i.e. the therapeutic gene expression at the protein level, and the possibility of increasing the level of protein expression by gene therapy DNA vector based on gene therapy DNA vector VTvafl7 carrying the VIP therapeutic gene.

The following elements are indicated in FIG. 9:
culture A—conditioned medium from HBdSMC human urinary bladder smooth muscle cells transfected with aqueous dendrimer solution without DNA vector (reference),
culture B—conditioned medium from HBdSMC human urinary bladder smooth muscle cells transfected with DNA vector VTvafl7,
culture C—conditioned medium from HBdSMC human urinary bladder smooth muscle cells transfected with DNA vector VTvafl7-VIP.

FIG. 10 shows the plot of KCNMA1 protein concentration in the cell lysate of HBdSMC human urinary bladder smooth muscle cells (ATCC PCS-420-012) after transfection of these cells with gene therapy DNA vector VTvafl7-KCNMA1 in order to assess the functional activity, i.e. the therapeutic gene expression at the protein level, and the possibility of increasing the level of protein expression by gene therapy DNA vector based on gene therapy DNA vector VTvafl7 carrying the KCNMA1 therapeutic gene.

The following elements are indicated in FIG. 10:
culture A—HBdSMC human urinary bladder smooth muscle cells transfected with aqueous dendrimer solution without DNA vector (reference),
culture B—HBdSMC human urinary bladder smooth muscle cells transfected with DNA vector VTvafl7,
culture C—HBdSMC human urinary bladder smooth muscle cells transfected with DNA vector VTvafl7-KCNMA1.

FIG. 11 shows the plot of CGRP protein concentration in the cell lysate of primary corpus cavernosum penis smooth muscle cells after transfection of these cells with gene therapy DNA vector VTvafl7-CGRP in order to assess the functional activity, i.e. the therapeutic gene expression at the protein level, and the possibility of increasing the level of protein expression by gene therapy DNA vector based on gene therapy DNA vector VTvafl7 carrying the CGRP therapeutic gene.

The following elements are indicated in FIG. 11:
culture A—human primary corpus cavernosum penis smooth muscle cells transfected with aqueous dendrimer solution without DNA vector (reference),
culture B—human primary corpus cavernosum penis smooth muscle cells transfected with DNA vector VTvafl7,
culture C—human primary corpus cavernosum penis smooth muscle cells transfected with DNA vector VTvafl7-CGRP.

FIG. 12 shows the plot of NOS2 protein concentration in the skin biopsy specimens of three patients after injection of gene therapy DNA vector VTvafl7-NOS2 into the skin of these patients in order to assess the functional activity, i.e. the expression of the therapeutic gene at the protein level, and the possibility of increasing the level of protein expression using gene therapy DNA vector based on gene therapy vector VTvafl7 carrying the NOS2 therapeutic gene.

The following elements are indicated in FIG. 12:
P1I—patient P1 skin biopsy in the region of injection of gene therapy DNA vector VTvafl7-NOS2,
P1II—patient P1 skin biopsy in the region of injection of gene therapy DNA vector VTvafl7 (placebo),
P1III—patient P1 skin biopsy from intact site,
P2I—patient P2 skin biopsy in the region of injection of gene therapy DNA vector VTvafl7-NOS2,
P2II—patient P2 skin biopsy in the region of injection of gene therapy DNA vector VTvafl7 (placebo),
P2II—patient P2 skin biopsy from intact site,
P3I—patient P3 skin biopsy in the region of injection of gene therapy DNA vector VTvafl7-NOS2,
P3II—patient P3 skin biopsy in the region of injection of gene therapy DNA vector VTvafl7 (placebo),
P3III—patient P3 skin biopsy from intact site.

FIG. 13 shows the plot of NOS3 protein concentration in the gastrocnemius muscle biopsy specimens of three patients after injection of gene therapy DNA vector VTvafl7-NOS3 into the gastrocnemius muscle of these patients in order to assess the functional activity, i.e. the therapeutic gene expression at the protein level, and the possibility of increasing the level of protein expression using gene therapy DNA vector based on gene therapy vector VTvafl7 carrying the NOS3 therapeutic gene.

The following elements are indicated in FIG. 13:
P1I—patient P1 gastrocnemius muscle biopsy in the region of injection of gene therapy DNA vector VTvafl7-NOS3,
P1II—patient P1 gastrocnemius muscle biopsy in the region of injection of gene therapy DNA vector VTvafl7 (placebo),
P1III—patient P1 gastrocnemius muscle biopsy from intact site,
P2I—patient P2 gastrocnemius muscle biopsy in the region of injection of gene therapy DNA vector VTvafl7-NOS3,
P2II—patient P2 gastrocnemius muscle biopsy in the region of injection of gene therapy DNA vector VTvafl7 (placebo),
P2II—patient P2 gastrocnemius muscle biopsy from intact site,
P3I—patient P3 gastrocnemius muscle biopsy in the region of injection of gene therapy DNA vector VTvafl7-NOS3,
P3II—patient P3 gastrocnemius muscle biopsy in the region of injection of gene therapy DNA vector VTvafl7 (placebo),
P3II—patient P3 gastrocnemius muscle biopsy from intact site.

FIG. 14 shows the plot of KCNMA1 protein concentration in the biopsy samples of patient's corpus cavernosum penis upon intracavernous injection of gene therapy DNA vector VTvafl7-KCNMA1 to the corpus cavernosum penis in order to demonstrate the method of use by intracavernous injection of gene therapy DNA vector.

The following elements are indicated in FIG. 14:
P1I—corpus cavernosum penis biopsy from the region of injection of gene therapy DNA vector VTvafl7-KCNMA1,
P1II—corpus cavernosum penis biopsy from the region of injection of gene therapy DNA vector VTvafl7 (placebo),
P1III—corpus cavernosum penis biopsy of patients from the region not subjected to any manipulation.

FIG. 15 shows the plot of NOS2 protein concentration in human skin biopsy samples after subcutaneous injection of autologous fibroblast cell culture transfected with the gene therapy DNA vector VTvafl7-NOS2 in order to demonstrate the method of use by injecting autologous cells transfected with the gene therapy DNA vector VTvafl7-NOS2.

The following elements are indicated in FIG. 15:
P1A—patient P1 skin biopsy in the region of injection of autologous fibroblast culture of the patient transfected with gene therapy DNA vector VTvafl7-NOS2,
P1B—patient P1 skin biopsy in the region of injection of autologous fibroblasts of the patient transfected with gene therapy DNA vector VTvafl7,
P1C—patient P1 skin biopsy from intact site.

FIG. 16 shows diagrams of cDNA amplicon accumulation of the KCNMA1 therapeutic gene in BAOSMC bovine aortic smooth muscle cells (Genlantis) before and 48 hours after transfection of these cells with the DNA vector VTvafl7-KCNMA1 in order to demonstrate the method of use by introducing the gene therapy DNA vector in animal cages.

The following curves of accumulation of amplicons during the reaction are shown in FIG. 16 corresponding to:
1—cDNA of KCNMA1 gene in bovine aortic smooth muscle cells BAOSMC before transfection with gene therapy DNA vector VTvafl7-KCNMA1,
2—cDNA of KCNMA1 gene in bovine aortic smooth muscle cells BAOSMC after transfection with gene therapy DNA vector VTvafl7-KCNMA1,
3—cDNA of B2M gene in bovine aortic smooth muscle cells BAOSMC before transfection with gene therapy DNA vector VTvafl7-KCNMA1,
4—cDNA of B2M gene in BAOSMC bovine aortic smooth muscle cells after transfection with gene therapy DNA vector VTvafl7-KCNMA1.

Bull/cow actin gene (ACT) listed in the GenBank database under number AH001130.2 was used as a reference gene.

### EMBODIMENT OF THE INVENTION

Gene therapy DNA vectors carrying the human therapeutic genes designed to increase the expression level of these therapeutic genes in human and animal tissues were constructed based on 3165 bp DNA vector VTvafl7. The method of production of each gene therapy DNA vector carrying human therapeutic genes involves cloning of the protein coding sequence of the therapeutic gene to the polylinker of the gene therapy DNA vector VTvafl7 selected from the group of the following genes: human NOS2, NOS3, VIP, KCNMA1, and CGRP gene. It is known that the ability of DNA vectors to penetrate into eukaryotic cells is due mainly to the vector size. DNA vectors with the smallest size have higher penetration capability. Thus, the absence of elements in the vector that bear no functional load, but at the same time increase the vector DNA size is preferred. These features of DNA vectors were taken into account during the production of gene therapy DNA vectors based on gene therapy DNA vector VTvafl7 carrying the therapeutic gene selected from the group of NOS2, NOS3, VIP, KCNMA1, and CGRP genes with no large non-functional sequences and antibiotic resistance genes in the vector, which, in addition to technological advantages and safe use, allowed for the significant reduction of size of the produced gene therapy DNA vector VTvafl7 carrying the therapeutic gene selected from the group of NOS2, NOS3, VIP, KCNMA1, and CGRP genes. Thus, the ability of the obtained gene therapy DNA vector to penetrate into eukaryotic cells is due to its small length.

Each of the following gene therapy DNA vectors: VTvafl7-NOS2, or VTvafl7-NOS3, or VTvafl7-VIP, or VTvafl7-KCNMA1, or VTvafl7-CGRP was produced as follows: the coding region of the therapeutic gene NOS2, or NOS3, or VIP, or KCNMA1, or CGRP genes was cloned to gene therapy DNA vector VTvafl7, and gene therapy DNA vector VTvafl7-NOS2, SEQ ID No. 1, or VTvafl7-NOS3, SEQ ID No. 2, or VTvafl7-VIP, SEQ ID No. 3, or VTvafl7-KCNMA1, SEQ ID No. 4, or VTvafl7-CGRP, SEQ ID No. 5, respectively, was obtained. The coding region of NOS2 gene (3466 bp), or NOS3 gene (3615 bp), or VIP gene (511 bp), or KCNMA1 gene (3578 bp), or CGRP gene (454 bp) was produced by extracting total RNA from the biological normal tissue sample. The reverse transcription reaction was used for the synthesis of the first chain cDNA of human NOS2, NOS3, VIP, KCNMA1, and CGRP genes. Amplification was performed using oligonucleotides produced for this purpose by the chemical synthesis method. The amplification product was cleaved by specific restriction endonucleases taking into account the optimal procedure for further cloning, and cloning to the gene therapy DNA vector VTvafl7 was performed by SalI, KpnI, BamHI, EcoRI, HindIII restriction sites located in the VTvafl7 vector polylinker. The selection of restriction sites was carried out in such a way that the cloned fragment entered the reading frame of expression cassette of the vector VTvafl7, while the protein coding sequence did not contain restriction sites for the selected endonucleases. Experts in this field realise that the methodological implementation of gene therapy DNA vector VTvafl7-NOS2, or VTvafl7-NOS3, or VTvafl7-VIP, or VTvafl7-KCNMA1, or VTvafl7-CGRP production can vary within the framework of the selection of known methods of molecular gene cloning and these methods are included in the scope of this invention. For example, different oligonucleotide sequences can be used to amplify NOS2, or NOS3, or VIP, or KCNMA1, or CGRP gene, different restriction endonucleases or laboratory techniques, such as ligation independent cloning of genes.

Gene therapy DNA vector VTvafl7-NOS2, or VTvafl7-NOS3, or VTvafl7-VIP, or VTvafl7-KCNMA1, or VTvafl7-CGRP has the nucleotide sequence SEQ ID No. 1, or SEQ ID No. 2, or SEQ ID No. 3, or SEQ ID No. 4, or SEQ ID No. 5, respectively. At the same time, degeneracy of genetic code is known to the experts in this field and means that the scope of this invention also includes variants of nucleotide sequences differing by insertion, deletion, or replacement of nucleotides that do not result in a change in the polypeptide sequence encoded by the therapeutic gene, and/or do not result in a loss of functional activity of the regulatory elements of VTvafl7 vector. At the same time, genetic polymorphism is known to the experts in this field and means that the scope of this invention also includes variants of nucleotide sequences of genes from the group of NOS2, NOS3, VIP, KCNMA1, or CGRP genes that also encode different variants of the amino acid sequences of NOS2, NOS3, VIP, KCNMA1, or CGRP proteins that do not differ from those listed in their functional activity under physiological conditions.

The ability to penetrate into eukaryotic cells and express functional activity, i.e. the ability to express the therapeutic gene of the obtained gene therapy DNA vector VTvafl7-NOS2, or VTvafl7-NOS3, or VTvafl7-VIP, or VTvafl7-KCNMA1, or VTvafl7-CGRP is confirmed by injecting the obtained vector into eukaryotic cells and subsequent analysis of the expression of specific mRNA and/or protein product of the therapeutic gene. The presence of specific mRNA in cells into which the gene therapy DNA vector VTvafl7-NOS2, or VTvafl7-NOS3, or VTvafl7-VIP, or VTvafl7-KCNMA1, or VTvafl7-CGRP was injected shows the ability of the obtained vector to both penetrate into eukaryotic cells and express mRNA of the therapeutic gene. Furthermore, it is known to the experts in this field that the presence of mRNA gene is a mandatory condition, but not an evidence of the translation of protein encoded by the therapeutic gene. Therefore, in order to confirm properties of the gene therapy DNA vector VTvafl7-NOS2, or VTvafl7-NOS3, or VTvafl7-VIP, or VTvafl7-KCNMA1, or VTvafl7-CGRP to express the therapeutic gene at the protein level in eukaryotic cells into which the gene therapy DNA vector was injected, analysis of the concentration of proteins encoded by the therapeutic genes was carried out using immunological methods. The presence of NOS2, or NOS3, or VIP, or KCNMA1, or CGRP protein confirms the efficiency of expression of therapeutic genes in eukaryotic cells and the possibility of increasing the protein concentration using the gene therapy DNA vector based on gene therapy DNA vector VTvafl7 carrying the therapeutic gene selected from the group of NOS2, NOS3, VIP, KCNMA1, and CGRP genes. To confirm the efficiency of the produced gene therapy DNA vector VTvafl7-NOS2 carrying the therapeutic gene, namely the NOS2 gene, gene therapy DNA vector VTvafl7-NOS3 carrying the therapeutic gene, namely the NOS3 gene, gene therapy DNA vector VTvafl7-VIP carrying the therapeutic gene, namely the VIP gene, gene therapy DNA vector VTvafl7-KCNMA1 carrying the therapeutic gene, namely the KCNMA1 gene, gene therapy DNA vector VTvafl7-CGRP carrying the therapeutic gene, namely the CGRP gene, the following methods were used:
A) real-time PCR, i.e. change in mRNA accumulation of therapeutic genes in human and animal cell lysate after transfection of different human and animal cell lines with gene therapy DNA vectors,
B) Enzyme-linked immunosorbent assay, i.e. change in the quantitative level of therapeutic proteins in the human cell lysate after transfection of different human cell lines with gene therapy DNA vectors,
C) Enzyme-linked immunosorbent assay, i.e. change in the quantitative level of therapeutic proteins in the supernatant of human and animals tissue biopsy specimens after the injection of gene therapy DNA vectors into these tissues,
D) Enzyme-linked immunosorbent assay, i.e. change in the quantitative level of therapeutic proteins in the supernatant of human tissue biopsies after the injection of these tissues with autologous cells of this human transfected with gene therapy DNA vectors.

In order to confirm the practicability of use of the constructed gene therapy DNA vector VTvafl7-NOS2 carrying the therapeutic gene, namely the NOS2 gene, gene therapy DNA vector VTvafl7-NOS3 carrying the therapeutic gene, namely the NOS3 gene, gene therapy DNA vector VTvafl7-VIP carrying the therapeutic gene, namely the VIP gene, gene therapy DNA vector VTvafl7-KCNMA1 carrying the therapeutic gene, namely the KCNMA1 gene, gene therapy DNA vector VTvafl7-CGRP carrying the therapeutic gene, namely the CGRP gene, the following was performed:
A) transfection of different human and animal cell lines with gene therapy DNA vectors,
B) injection of gene therapy DNA vectors into different human and animal tissues,
C) injection of autologous cells transfected with gene therapy DNA vectors into human tissues.

These methods of use lack potential risks for gene therapy of humans and animals due to the absence of regulatory elements in the gene therapy DNA vector that constitute the nucleotide sequences of viral genomes and absence of antibiotic resistance genes in the gene therapy DNA vector as confirmed by the lack of regions homologous to the viral genomes and antibiotic resistance genes in the nucleotide sequences of gene therapy DNA vector VTvafl7-NOS2, or gene therapy DNA vector VTvafl7-NOS3, or gene therapy DNA vector VTvafl7-VIP, or gene therapy DNA vector VTvafl7-KCNMA1, or gene therapy DNA vector VTvafl7-CGRP (SEQ ID No. 1, or SEQ ID No. 2, or SEQ ID No. 3, or SEQ ID No. 4, or SEQ ID No. 5, respectively).

It is known to the experts in this field that antibiotic resistance genes in the gene therapy DNA vectors are used to obtain these vectors in preparative quantities by increasing bacterial biomass in a nutrient medium containing a selective antibiotic. Within the framework of this invention, in order to ensure the safe use of gene therapy DNA vector VTvafl7 carrying NOS2, or NOS3, or VIP or KCNMA1, or CGRP therapeutic genes, the use of selective nutrient media containing an antibiotic is not possible. A method for obtaining strains for production of these gene therapy vectors based on Escherichia coli strain SCS110-AF is proposed as a technological solution for obtaining the gene therapy DNA vector VTvafl7 carrying a therapeutic gene selected from the group of NOS2, NOS3, VIP, KCNMA1, and CGRP genes in order to scale up the production of gene therapy vectors to an industrial scale. The method of Escherichia coli strain SCS110-AF/VTvafl7-NOS2, or Escherichia coli strain SCS110-AF/VTvafl7-NOS3, or Escherichia coli strain SCS110-AF/VTvafl7-VIP, or Escherichia coli strain SCS110-AF/VTvafl7-KCNMA1, or Escherichia coli strain SCS110-AF/VTvafl7-CGRP production involves production of competent cells of Escherichia coli strain SCS110-AF with the injection of gene therapy DNA vector VTvafl7-NOS2, or DNA vector VTvafl7-NOS3, or DNA vector VTvafl7-VIP, or DNA vector VTvafl7-KCNMA1, or DNA vector VTvafl7-CGRP into these cells, respectively, using transformation (electroporation) methods widely known to experts in this field. The obtained Escherichia coli strain SCS110-AF/VTvafl7-NOS2, or Escherichia coli strain SCS110-AF/VTvafl7-NOS3, or Escherichia coli strain SCS110-AF/VTvafl7-VIP, or Escherichia coli strain SCS110-AF/VTvafl7-KCNMA1, or Escherichia coli strain SCS110-AF/VTvafl7-CGRP is used to produce the gene therapy DNA vector VTvafl7-NOS2, or VTvafl7-NOS3, or VTvafl7-VIP, or VTvafl7-KCNMA1, or VTvafl7-CGRP, respectively, allowing the use of antibiotic-free media.

To confirm the producibility and constructability and scale up of the production of gene therapy DNA vector VTvafl7-NOS2 carrying the therapeutic gene, namely the NOS2 gene, gene therapy DNA vector VTvafl7-NOS3 carrying the therapeutic gene, namely the NOS3 gene, gene therapy DNA vector VTvafl7-VIP carrying the therapeutic gene, namely the VIP gene, gene therapy DNA vector VTvafl7-KCNMA1 carrying the therapeutic gene, namely the KCNMAI gene, gene therapy DNA vector VTvafl7-CGRP carrying the therapeutic gene, namely the CGRP gene, to an industrial scale, the fermentation on an industrial scale of Escherichia coli strain SCS110-AF/VTvafl7-NOS2, or Escherichia coli strain SCS110-AF/VTvafl7-NOS3, or Escherichia coli strain SCS110-AF/VTvafl7-VIP, or Escherichia coli strain SCS110-AF/VTvafl7-KCNMA1, or Escherichia coli strain SCS110-AF/VTvafl7-CGRP each containing gene therapy DNA vector VTvafl7 carrying the therapeutic gene, namely NOS2, or NOS3, or VIP, or KCNMA1, or CGRP gene, was performed.

The method of scaling the production of bacterial mass to an industrial scale for the isolation of gene therapy DNA vector VTvafl7 carrying the therapeutic gene selected from the group of NOS2, NOS3, VIP, KCNMA1, and CGRP genes involves incubation of the seed culture of Escherichia coli strain SCS110-AF/VTvafl7-NOS2, or Escherichia coli strain SCS110-AF/VTvafl7-NOS3, or Escherichia coli strain SCS110-AF/VTvafl7-VIP, or Escherichia coli strain SCS110-AF/VTvafl7-KCNMA1, or Escherichia coli strain SCS110-AF/VTvafl7-CGRP in the antibiotic-free nutrient medium that provides suitable biomass accumulation dynamics. Upon reaching a sufficient amount of biomass in the logarithmic phase, the bacterial culture is transferred to an industrial fermenter and then grown to a stationary phase, then the fraction containing the therapeutic DNA product, i.e. the gene therapy DNA vector VTvafl7-NOS2, or gene therapy DNA vector VTvafl7-NOS3, or gene therapy DNA vector VTvafl7-VIP, or gene therapy DNA vector VTvafl7-KCNMA1, or gene therapy DNA vector VTvafl7-CGRP is extracted, multi-stage filtered, and purified by chromatographic methods. It is known to the experts in this field that culture conditions of strains, composition of nutrient media (except for antibiotic-free), equipment used, and DNA purification methods may vary within the framework of standard operating procedures depending on the particular production line, but known approaches to scaling, industrial production, and purification of DNA vectors using Escherichia coli strain SCS110-AF/VTvafl7-NOS2, or Escherichia coli strain SCS110-AF/VTvafl7-NOS3, or Escherichia coli strain SCS110-AF/VTvafl7-VIP, or Escherichia coli strain SCS110-AF/VTvafl7-KCNMA1, or Escherichia coli strain SCS110-AF/VTvafl7-CGRP fall within the scope of this invention.

The described disclosure of the invention is illustrated by examples of the embodiment of this invention.

The essence of the invention is explained in the following examples.

### Example 1

Production of gene therapy DNA vector VTvafl7-NOS2 carrying the therapeutic gene, namely the NOS2 gene.

Gene therapy DNA vector VTvafl7-NOS2 was constructed by cloning the coding region of NOS2 gene (3466 bp) to a 3165 bp DNA vector VTvafl7 by SalI and KpnI restriction sites. The coding region of NOS2 gene (3466 bp) was obtained by isolating total RNA from the biological human tissue sample followed by reverse transcription reaction using commercial kit Mint-2 (Evrogen, Russia) and PCR amplification using the following oligonucleotides:
NOS2_F ATCGTCGACCACCATGGCCTGTCCTTGGAAATTTC,
NOS2_R CGGTACCTCAGAGCGCTGACATCTCCAGG
and commercially available kit Phusion® High-Fidelity DNA Polymerase (New England Biolabs, USA).

Gene therapy DNA vector VTvafl7 was constructed by consolidating six fragments of DNA derived from different sources:
(a) the origin of replication was produced by PCR amplification of a region of commercially available plasmid pBR322 with a point mutation,
(b) EF1a promoter region was produced by PCR amplification of a site of human genomic DNA,
(c) hGH-TA transcription terminator was produced by PCR amplification of a site of human genomic DNA,
(d) the RNA-OUT regulatory site of transposon Tn10 was synthesised from oligonucleotides,
(e) kanamycin resistance gene was produced by PCR amplification of a site of commercially available plasmid pET-28,
(f) the polylinker was produced by annealing two synthetic oligonucleotides.

PCR amplification was performed using the commercially available kit Phusion® High-Fidelity DNA Polymerase (New England Biolabs, USA) as per the manufacturer's instructions. The fragments have overlapping regions allowing for their consolidation with subsequent PCR amplification. Fragments (a) and (b) were consolidated using oligonucleotides Ori-F and EF1-R, and fragments (c), (d), and (e) were consolidated using oligonucleotides hGH-F and Kan-R. Afterwards, the produced fragments were consolidated by restriction with subsequent ligation by sites BamHI and NcoI. This resulted in a plasmid still devoid of the polylinker. To add it, the plasmid was cleaved by BamHI and EcoRI sites followed by ligation with fragment (f). Therefore, a 4182 bp vector was constructed carrying the kanamycin resistance gene flanked by SpeI restriction sites. Then this gene was cleaved by SpeI restriction sites and the remaining fragment was ligated to itself. This resulted in a 3165 bp gene therapy DNA vector VTvafl7 that is recombinant and allows for antibiotic-free selection.

The amplification product of the coding region of NOS2 gene and DNA vector VTvafl7 was cleaved by restriction endonucleases SalI and KpnI (New England Biolabs, USA).

This resulted in a 6625 bp DNA vector VTvafl7-NOS2 with the nucleotide sequence SEQ ID No. 1 and general structure shown in FIG. 1.

### Example 2

Production of gene therapy DNA vector VTvafl7-NOS3 carrying the therapeutic gene, namely the NOS3 gene.

Gene therapy DNA vector VTvafl7-NOS3 was constructed by cloning the coding region of NOS3 gene (3615 bp) to a 3165 bp DNA vector VTvafl7 by HindIIII and EcoRI restriction sites. The coding region of NOS3 gene (3615 bp) was obtained by isolating total RNA from the biological human tissue sample followed by reverse transcription reaction using commercial kit Mint-2 (Evrogen, Russia) and PCR amplification using the following oligonucleotides:

NOS3_F
GACAAGCTTCCACCATGGGCAACTTGAAGAG,
NOS3_R
GGAATTCAGGGGCTGTTGGTGTCTGAGCCG

and commercially available kit Phusion® High-Fidelity DNA Polymerase (New England Biolabs, USA); amplification product and DNA vector VTvafl7 were cleaved by restriction endonucleases HindIIII and EcoRI (New England Biolabs, USA).

This resulted in a 6774 bp DNA vector VTvafl7-NOS3 with the nucleotide sequence SEQ ID No. 2 and general structure shown in FIG. 1.

Gene therapy DNA vector VTvafl7 was constructed as described in Example 1.

### Example 3

Production of gene therapy DNA vector VTvafl7-VIP carrying the therapeutic gene, namely the human VIP gene.

Gene therapy DNA vector VTvafl7-VIP was constructed by cloning the coding region of VIP gene (511 bp) to a 3165 bp DNA vector VTvafl7 by BamHI and EcoRI restriction sites. The coding region of VIP gene (511 bp) was obtained by isolating total RNA from the biological human tissue sample followed by reverse transcription reaction using commercial kit Mint-2 (Evrogen, Russia) and PCR amplification using the following oligonucleotides:

VIP_F
AGGATCCACCATGGACACCAGAAATAAGGCCCAG,
VIP_R
GGAATTCATTTTTCTAACTCTTCTGGAAAG

and commercially available kit Phusion® High-Fidelity DNA Polymerase (New England Biolabs, USA); amplification product and DNA vector VTvafl7 were cleaved by restriction endonucleases BamHI and EcoRI (New England Biolabs, USA).

This resulted in a 3652 bp DNA vector VTvafl7-VIP with the nucleotide sequence SEQ ID No. 3 and general structure shown in FIG. 1.

Gene therapy DNA vector VTvafl7 was constructed as described in Example 1.

### Example 4

Production of gene therapy DNA vector VTvafl7-KCNMA1 carrying the therapeutic gene, namely the KCNMA1 gene.

Gene therapy DNA vector VTvafl7-KCNMA1 was constructed by cloning the coding region of KCNMA1 gene (3578 bp) to a 3165 bp DNA vector VTvafl7 by BamHI and EcoRI restriction sites. The coding region of KCNMA1 gene (3578 bp) was obtained by isolating total RNA from the biological human tissue sample followed by reverse transcription reaction using commercial kit Mint-2 (Evrogen) and PCR amplification using the following oligonucleotides:

KCNMA1_F
AGGATCCGGTACCGAGGAGATCTGCCGCCGCGATCGCCATG,
KCNMA1_R
ACCAAGCTTATCTGTAAACCATTTCTTTTCTG

and commercially available kit Phusion® High-Fidelity DNA Polymerase (New England Biolabs, USA); amplification product and DNA vector VTvafl7 were cleaved by restriction endonucleases BamHI and EcoRI (New England Biolabs).

This resulted in a 6731 bp DNA vector VTvafl7-KCNMA1 with the nucleotide sequence SEQ ID No. 4 and general structure shown in FIG. 1.

Gene therapy DNA vector VTvafl7 was constructed as described in Example 1.

### Example 5

Production of gene therapy DNA vector VTvafl7-CGRP carrying the therapeutic gene, namely the CGRP gene.

Gene therapy DNA vector VTvafl7-CGRP was constructed by cloning the coding region of CGRP gene (454 bp) to a 3165 bp DNA vector VTvafl7 by BamHI and EcoRI restriction sites. The coding region of CGRP gene (454 bp) was obtained by isolating total RNA from the biological human tissue sample followed by reverse transcription reaction using commercial kit Mint-2 (Evrogen, Russia) and PCR amplification using the following oligonucleotides:

CGRP_F
AGGATCCGGACGTCATGGAAGTGAAGGATGCCAATT,
CGRP_R
GGAATTCCTATGCTGGGTCCTCTTCGTCCATTG

and commercially available kit Phusion® High-Fidelity DNA Polymerase (New England Biolabs, USA); amplification product and DNA vector VTvafl7 were cleaved by restriction endonucleases BamHI and EcoRI (New England Biolabs).

This resulted in a 3595 bp DNA vector VTvafl7-CGRP with the nucleotide sequence SEQ ID No. 5 and general structure shown in FIG. 1.

Gene therapy DNA vector VTvafl7 was constructed as described in Example 1.

### Example 6

Proof of the ability of gene therapy DNA vector VTvafl7-NOS2 carrying the therapeutic gene, namely NOS2 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the NOS2 therapeutic gene were assessed in HBdSMC primary human urinary bladder smooth muscle cells (ATCC PCS-420-012) 48 hours after their transfection with gene therapy DNA vector VTvafl7-NOS2 carrying the human NOS2 gene. The amount of mRNA was determined by the dynamics of accumulation of cDNA amplicons in the real-time PCR.

HBdSMC primary human urinary bladder smooth muscle cell culture was grown in the medium with growth additives prepared using the Vascular Smooth Muscle Cell GroCGRPh Kit (ATCC® PCS-100-042™) under standard conditions (37° C., 5% CO2). To achieve 90% confluence, 24 hours before the transfection procedure, the cells were seeded into a 24-well plate in the quantity of 5×104 cells per well. Transfection with gene therapy DNA vector VTvafl7-NOS2 expressing the human NOS2 gene was performed using Lipofectamine 3000 (ThermoFisher Scientific, USA). In test tube 1, 1 μl of DNA vector VTvafl7-NOS2 solution (concentration 500 ng/μl) and 1 μl of reagent P3000 was added to 25 μl of medium Opti-MEM (Gibco, USA). The preparation was mixed by gentle shaking. In test tube 2, 1 μl of Lipofectamine 3000 solution was added to 25 μl of medium Opti-MEM (Gibco, USA). The preparation was mixed by gentle shaking. The contents from test tube 1 were added to the contents of test tube 2, and the mixture was incubated at room temperature for 5 minutes. The resulting solution was added dropwise to the cells in the volume of 40 μl.

Cells transfected with gene therapy DNA vector VTvafl7 devoid of the inserted therapeutic gene were used as a reference. Reference vector VTvafl7 for transfection was prepared as described above.

Total RNA from transfected cells was isolated using Trizol Reagent (Invitrogen, USA). 1 ml of Trizol Reagent was added to the well with cells and homogenised and heated for 5 minutes at 65° C. Then the sample was centrifuged at 14,000 g for 10 minutes and heated again for 10 minutes at 65° C. Then 200 μl of chloroform was added, and the mixture was gently stirred and centrifuged at 14,000 g for 10 minutes. Then the water phase was isolated and mixed with 1/10 of the volume of 3M sodium acetate, pH 5.2, and an equal volume of isopropyl alcohol. The sample was incubated at −20° C. for 10 minutes and then centrifuged at 14,000 g for 10 minutes. The precipitated RNA were rinsed in 1 ml of 70% ethyl alcohol, air-dried and dissolved in 10 μl of RNase-free water. The level of NOS2 mRNA expression after transfection was determined by assessing the dynamics of the accumulation of cDNA amplicons by real-time PCR. For the production and amplification of cDNA specific for the human NOS2 gene, the following NOS2_SF and NOS2_SR oligonucleotides were used:

NOS2_SF
AACGTGTTCACCATGAGGCT,
NOS2_SR
CTCTCAGGCTCTTCTGTGGC.

The length of amplification product is 377 bp.

Reverse transcription reaction and PCR amplification was performed using SYBR GreenQuantitect RT-PCR Kit (Qiagen, USA) for real-time PCR. The reaction was carried out in a volume of 20 μl, containing: 25 μl of QuantiTect SYBR Green RT-PCR Master Mix, 2.5 mM of magnesium chloride, 0.5 μM of each primer, and 5 μl of RNA. For the reaction, CFX96 amplifier (Bio-Rad, USA) was used under the following conditions: 1 cycle of reverse transcription at 42° C. for 30 minutes, denaturation at 98° C. for 15 minutes, followed by 40 cycles comprising denaturation at 94° C. for 15 s, annealing of primers at 60° C. for 30 s and elongation at 72° C. for 30 s. B2M (beta-2-microglobuline) gene listed in the GenBank database under number NM 004048.2 was used as a reference gene. Positive control included amplicons from PCR on matrices represented by plasmids in known concentrations containing cDNA sequences of NOS2 and B2M genes. Negative control included deionised water. Real-time quantification of the dynamics of accumulation of cDNA amplicons of NOS2 and B2M genes was conducted using the Bio-Rad CFX Manager 2.1 software (Bio-Rad, USA). Diagrams resulting from the assay are shown in FIG. 2.

FIG. 2 shows that cDNA amplicons were accumulated faster than in reference samples of cells as a result of transfection of HBdSMc human urinary bladder smooth muscle cell culture with gene therapy DNA vector VTvafl7-NOS2, which indicates higher mRNA level of human NOS2 gene in the cells after transfection with gene therapy DNA vector VTvafl7-NOS2 and confirms the ability of the vector to penetrate eukaryotic cells and express the NOS2 gene at the mRNA level. The presented results also confirm the practicability of use of gene therapy DNA vector VTvafl7-NOS2 in order to increase the expression level of NOS2 gene in eukaryotic cells.

### Example 7

Proof of the ability of gene therapy DNA vector VTvafl7-NOS3 carrying the therapeutic gene, namely NOS3 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the NOS3 therapeutic gene were assessed in T/G HA-VSMC primary human aortic smooth muscle cell culture (ATCC CRL-1999™) 48 hours after its transfection with gene therapy DNA vector VTvafl7-NOS3 carrying the human NOS3 gene. The amount of mRNA was determined by the dynamics of accumulation of cDNA amplicons in the real-time PCR.

T/G HA-VSMC primary human aortic smooth muscle cell culture was grown in F-12K Medium (ATCC) with the addition of 0.05 mg/ml ascorbic acid, 0.01 mg/ml insulin, 0.01 mg/ml transferrin, 10 ng/ml sodium selenite, 0.03 mg/ml Endothelial Cell Growth Supplement (ECGS), 10% fetal bovine serum at 37° C. in the presence of 5% CO2. To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvafl7-NOS3 expressing the human NOS3 gene was performed according to the procedure described in Example 6. T/GHA-VSMC primary aortic smooth muscle cells transfected with the gene therapy DNA vector VTvafl7 devoid of the therapeutic gene were used as a reference. RNA isolation, reverse transcription reaction, and real-time PCR were performed as described in Example 6, except for oligonucleotides with sequences different from Example 6. For the amplification of cDNA specific for the human NOS3 gene, the following NOS3_SF and NOS3_SR oligonucleotides were used:

NOS3_SF
GACCCACTGGTGTCCTCTTG,
NOS3_SR
CTCCGTTTGGGGCTGAAGAT

The length of amplification product is 329 bp.

B2M (beta-2-microglobuline) gene listed in the GenBank database under number NM 004048.2 was used as a reference gene. Positive control included amplicons from PCR on matrices represented by plasmids in known concentrations containing cDNA sequences of NOS3 and B2M genes. Negative control included deionised water. Real-time quantification of the PCR products, i.e. NOS3 and B2M gene cDNAs obtained by amplification, was conducted using the Bio-Rad CFX Manager 2.1 software (Bio-Rad, USA). Diagrams resulting from the assay are shown in FIG. 3.

FIG. 3 shows that cDNA amplicons were accumulated faster than in reference samples of cells as a result of transfection of T/GHA-VSMC primary aortic smooth muscle cell culture with gene therapy DNA vector VTvafl7-NOS3, which indicates higher mRNA level of human NOS3 gene in the cells after transfection with gene therapy DNA vector VTvafl7-NOS3 and confirms the ability of the vector to penetrate eukaryotic cells and express the NOS3 gene at the mRNA level. The presented results also confirm the practicability of use of gene therapy DNA vector VTvafl7-NOS3 in order to increase the expression level of NOS3 gene in eukaryotic cells.

### Example 8

Proof of the ability of gene therapy DNA vector VTvafl7-VIP carrying the therapeutic gene, namely VIP gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the VIP therapeutic gene were assessed in HBdSMC primary human urinary bladder smooth muscle cells (ATCC PCS-420-012) 48 hours after their transfection with gene therapy DNA vector VTvafl7-VIP carrying the human VIP gene. The amount of mRNA was determined by the dynamics of accumulation of cDNA amplicons in the real-time PCR.

HBdSMC primary human urinary bladder smooth muscle cell culture was grown in the medium with growth additives prepared using the Vascular Smooth Muscle Cell GroCGRPh Kit (ATCC® PCS-100-042™) under standard conditions (37° C., 5% CO2). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvafl7-VIP expressing the human VIP gene was performed according to the procedure described in Example 6. HBdSMc human primary bladder smooth muscle cells transfected with gene therapy DNA vector VTvafl7 devoid of the therapeutic gene were used as a reference. RNA isolation, reverse transcription reaction, and real-time PCR were performed as described in Example 6, except for oligonucleotides with sequences different from Example 6. For the amplification of cDNA specific for the human VIP gene, the following VIP SF and VIP_SR oligonucleotides were used:

VIP_SF
CCTTCTGCTCTCAGGTTGGG,
VIP_SR
CCCTCACTGCTCCTCTTTCC

The length of amplification product is 380 bp.

B2M (beta-2-microglobuline) gene listed in the GenBank database under number NM 004048.2 was used as a reference gene. Positive control included amplicons from PCR on matrices represented by plasmids in known concentrations containing cDNA sequences of VIP and B2M genes. Negative control included deionised water. Real-time quantification of the PCR products, i.e. VIP and B2M gene cDNAs obtained by amplification, was conducted using the Bio-Rad CFX Manager 2.1 software (Bio-Rad, USA). Diagrams resulting from the assay are shown in FIG. 4.

FIG. 4 shows that cDNA amplicons were accumulated faster than in reference samples of cells as a result of transfection of HBdSMc human urinary bladder smooth muscle cell culture with gene therapy DNA vector VTvafl7-VIP, which indicates higher mRNA level of human VIP gene in the cells after transfection with gene therapy DNA vector VTvafl7-VIP and confirms the ability of the vector to penetrate eukaryotic cells and express the VIP gene at the mRNA level. The presented results also confirm the practicability of use of gene therapy DNA vector VTvafl7-VIP in order to increase the expression level of VIP gene in eukaryotic cells.

### Example 9

Proof of the ability of gene therapy DNA vector VTvafl7-KCNMA1 carrying the therapeutic gene, namely KCNMA1 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the KCNMA1 therapeutic gene were assessed in primary human corpus cavernosum penis cell culture 48 hours after its transfection with gene therapy DNA vector VTvafl7-KCNMA1 carrying the human KCNMA1 gene. The amount of mRNA was determined by the dynamics of accumulation of cDNA amplicons in the real-time PCR.

Primary human corpus cavernosum penis cells were grown as follows. The patient's penile biopsy was taken from corpus cavernosum penis sites using the biopsy sampler MAGNUM (BARD, USA). The patient skin in the biopsy site was preliminarily rinsed with sterile saline and anaesthetised with a lidocaine solution. The biopsy sample size was ca. 10 mm3, and the weight was approximately 11 mg. The sample was placed in a buffer solution containing 0.05% trypsin (Gibco, USA) and 10 mM of EDTA. Cells were incubated with stirring in the magnetic stirrer at 37° C. Then the cell suspension was filtered using 100 μm pore size filters (Nalgen, USA), centrifuged for 10 minutes at 130 g, the precipitated cells were re-suspended in 15 ml of DMEM (Gibco, USA) containing 10% fetal bovine serum (Gibco, USA), 2 mM of glutamine, 10 μg/ml of gentamicin placed in 75 cm2 flask (Eppendorf), and incubated for 36-72 hours at 37° C. in the presence of 5% CO2. To achieve 90% confluence, 24 hours before the transfection procedure, the cells were seeded into a 24-well plate in the quantity of 5×104 cells per well.

Lipofectamine 3000 (ThermoFisher Scientific, USA) was used as a transfection reagent. The transfection with gene therapy DNA vector VTvafl7-KCNMA1 expressing the human KCNMA1 gene was performed according to the procedure described in Example 6. T HESCs immortalised human fibroblast cell culture transfected with gene therapy DNA vector VTvafl7 devoid of the therapeutic gene were used as a reference. RNA isolation, reverse transcription reaction, and real-time PCR were performed as described in Example 6, except for oligonucleotides with sequences different from Example 6. For the amplification of cDNA specific for the human KCNMA1 gene, the following KCNMAI_SF and KCNMAI_SR oligonucleotides were used:

KCNMA1_SF
GAGAGAGCCGAAGCCGAAAG,
KCNMA1_SR
ACCCTTGGGAATTAGCCTGC.

The length of amplification product is 825 bp.

B2M (beta-2-microglobuline) gene listed in the GenBank database under number NM 004048.2 was used as a reference gene. Positive control included amplicons from PCR on matrices represented by plasmids in known concentrations containing cDNA sequences of KCNMA1 and B2M genes. Negative control included deionised water. Real-time quantification of the PCR products, i.e. KCNMA1 and B2M gene cDNAs obtained by amplification, was conducted using the Bio-Rad CFX Manager 2.1 software (Bio-Rad, USA). Diagrams resulting from the assay are shown in FIG. 5.

FIG. 5 shows that cDNA amplicons were accumulated faster than in reference samples of cells as a result of transfection of human corpus cavernosum penis cell culture with gene therapy DNA vector VTvafl7-KCNMA1, which indicates higher mRNA level of human KCNMA1 gene in the cells after transfection with gene therapy DNA vector VTvafl7-KCNMA1 and confirms the ability of the vector to penetrate eukaryotic cells and express the KCNMA1 gene at the mRNA level. The presented results also confirm the practicability of use of gene therapy DNA vector VTvafl7-KCNMA1 in order to increase the expression level of KCNMA1 gene in eukaryotic cells.

### Example 10

Proof of the ability of gene therapy DNA vector VTvafl7-CGRP carrying the therapeutic gene, namely CGRP gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the CGRP therapeutic gene were assessed in HBdSMC primary human urinary bladder smooth muscle cells (ATCC PCS-420-012) 48 hours after their transfection with gene therapy DNA vector VTvafl7-CGRP carrying the human CGRP gene. The amount of mRNA was determined by the dynamics of accumulation of cDNA amplicons in the real-time PCR.

HBdSMC primary human urinary bladder smooth muscle cell culture was grown in the medium with growth additives prepared using the Vascular Smooth Muscle Cell GroCGRPh Kit (ATCC® PCS-100-042™) under standard conditions (37° C., 5% CO2). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvafl7-CGRP expressing the human CGRP gene was performed according to the procedure described in Example 6. HBdSMc human primary bladder smooth muscle cells transfected with gene therapy DNA vector VTvafl7 devoid of the therapeutic gene were used as a reference. RNA isolation, reverse transcription reaction, and real-time PCR were performed as described in Example 6, except for oligonucleotides with sequences different from Example 6. For the amplification of cDNA specific for the human CGRP gene, the following CGRP_SF and CGRP_SR oligonucleotides were used:

CGRP_SF
ACTGATCTGAAAGAGCAGCGT,
CGRP_SR
AGAATGCTGGTGACGGTGTG

The length of amplification product is 311 bp.

B2M (beta-2-microglobuline) gene listed in the GenBank database under number NM 004048.2 was used as a reference gene. Positive control included amplicons from PCR on matrices represented by plasmids in known concentrations containing cDNA sequences of CGRP and B2M genes. Negative control included deionised water. Real-time quantification of the PCR products, i.e. CGRP and B2M gene cDNAs obtained by amplification, was conducted using the Bio-Rad CFX Manager 2.1 software (Bio-Rad, USA). Diagrams resulting from the assay are shown in FIG. 6.

FIG. 6 shows that cDNA amplicons were accumulated faster than in reference samples of cells as a result of transfection of HBdSMc human urinary bladder smooth muscle cell culture with gene therapy DNA vector VTvafl7-CGRP, which indicates higher mRNA level of human CGRP gene in the cells after transfection with gene therapy DNA vector VTvafl7-CGRP and confirms the ability of the vector to penetrate eukaryotic cells and express the CGRP gene at the mRNA level. The presented results also confirm the practicability of use of gene therapy DNA vector VTvafl7-CGRP in order to increase the expression level of CGRP gene in eukaryotic cells.

### Example 11

Proof of the efficiency and practicability of use of gene therapy DNA vector VTvafl7-NOS2 carrying the NOS2 gene in order to increase the expression of NOS2 protein in mammalian cells.

The change in the NOS2 protein concentration in the cell lysate of HBdSMC primary human urinary bladder smooth muscle cell culture (ATCC PCS-420-012) was assessed after transfection of these cells with the DNA vector VTvafl7-NOS2 carrying the human NOS2 gene.

HBdSMC primary human urinary bladder smooth muscle cell culture was grown in the medium with growth additives prepared using the Vascular Smooth Muscle Cell GroCGRPh Kit (ATCC® PCS-100-042™) under standard conditions (37° C., 5% CO2). To achieve 90% confluence, 24 hours before the transfection procedure, the cells were seeded into a 24-well plate in the quantity of 5×104 cells per well. The 6th generation SuperFect Transfection Reagent (Qiagen, Germany) was used for transfection. The aqueous dendrimer solution without DNA vector (A) and DNA vector VTvafl7 devoid of cDNA of NOS2 gene (B) were used as a reference, and DNA vector VTvafl7-NOS2 carrying the human NOS2 gene was used as the transfected agent. The DNA-dendrimer complex was prepared according to the manufacturer's procedure (QIAGEN, SuperFect Transfection Reagent Handbook, 2002) with some modifications. For cell transfection in one well of a 24-well plate, McCoy's 5A Medium was added to 1 μg of DNA vector dissolved in TE buffer to a final volume of 60 μl, then 5 μl of SuperFect Transfection Reagent was added and gently mixed by pipetting five times. The complex was incubated at room temperature for 10-15 minutes. Then the culture medium was taken from the wells, the wells were rinsed with 1 ml of PBS buffer. 350 μl of McCoy's 5A Medium containing 10 μg/ml of gentamicin was added to the resulting complex, mixed gently, and added to the cells. The cells were incubated with the complexes for 2-3 hours at 37° C. in the presence of 5% CO2.

The medium was then removed carefully, and the live cell array was rinsed with 1 ml of PBS buffer. Then, McCoy's 5A Medium containing 10 μg/ml of gentamicin was added and incubated for 24-48 hours at 37° C. in the presence of 5% CO2.

After transfection, cells were rinsed three times with PBS, and then 1 ml of PBS was added to the cells and the cells were subjected to freezing/thawing three times. Then the suspension was centrifuged for 15 minutes at 15,000 rpm, and supernatant was collected and used for the quantification and assay of the therapeutic protein.

The NOS2 protein was assayed by enzyme-linked immunosorbent assay (ELISA) using the ELISA Kit for Nitric Oxide Synthase 2, Inducible (NOS2) (Cloud-Clone Corp. Cat. SEA837Hu, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of NOS2 protein was used. The sensitivity was at least 54 μg/ml (0.057 ng/ml), measurement range—from 156 μg/ml to 10000 μg/ml (0.156-10 ng/ml). R-3.0.2 was used for the statistical treatment of the results and data visualization (https://www.r-project.org/). Diagrams resulting from the assay are shown in FIG. 7.

FIG. 7 shows that the transfection of HBdSMc human urinary bladder smooth muscle cells with gene therapy DNA vector VTvafl7-NOS2 results in increased NOS2 protein concentration compared to reference samples, which confirms the ability of the vector to penetrate eukaryotic cells and express NOS2 gene at the protein level. The presented results also confirm the practicability of use of gene therapy DNA vector VTvafl7-NOS2 in order to increase the expression level of NOS2 gene in eukaryotic cells.

### Example 12

Proof of the efficiency and practicability of use of gene therapy DNA vector VTvafl7-NOS3 carrying the NOS3 gene in order to increase the expression of NOS3 protein in mammalian cells.

The change in the NOS3 protein concentration in the cell lysate of HBdSMC primary human urinary bladder smooth muscle cell culture (ATCC PCS-420-012) was assessed after transfection of these cells with the DNA vector VTvafl7-NOS3 carrying the human NOS3 gene.

HBdSMC primary human urinary bladder smooth muscle cell culture was grown in the medium with growth additives prepared using the Vascular Smooth Muscle Cell GroCGRPh Kit (ATCC® PCS-100-042™) under standard conditions (37° C., 5% CO2). To achieve 90% confluence, 24 hours before the transfection procedure, the cells were seeded into a 24-well plate in the quantity of 5×104 cells per well. The 6th generation SuperFect Transfection Reagent (Qiagen, Germany) was used for transfection. The aqueous dendrimer solution without DNA vector (A) and DNA vector VTvafl7 devoid of cDNA of NOS3 gene (B) were used as a reference, and DNA vector VTvafl7-NOS3 carrying the human NOS3 gene was used as the transfected agent. The DNA-dendrimer complex was prepared according to the manufacturer's procedure (QIAGEN, SuperFect Transfection Reagent Handbook, 2002) with some modifications. For cell transfection in one well of a 24-well plate, McCoy's 5A Medium was added to 1 μg of DNA vector dissolved in TE buffer to a final volume of 60 μl, then 5 μl of SuperFect Transfection Reagent was added and gently mixed by pipetting five times. The complex was incubated at room temperature for 10-15 minutes. Then the culture medium was taken from the wells, the wells were rinsed with 1 ml of PBS buffer. 350 μl of McCoy's 5A Medium containing 10 μg/ml of gentamicin was added to the resulting complex, mixed gently, and added to the cells. The cells were incubated with the complexes for 2-3 hours at 37° C. in the presence of 5% CO2.

The medium was then removed carefully, and the live cell array was rinsed with 1 ml of PBS buffer. Then, McCoy's 5A Medium containing 10 μg/ml of gentamicin was added and incubated for 24-48 hours at 37° C. in the presence of 5% CO2.

After transfection, cells were rinsed three times with PBS, and then 1 ml of PBS was added to the cells and the cells were subjected to freezing/thawing three times. Then the suspension was centrifuged for 15 minutes at 15,000 rpm, and supernatant was collected and used for the quantification and assay of the therapeutic protein.

The NOS3 protein was assayed by enzyme-linked immunosorbent assay (ELISA) using the ELISA Kit for Nitric Oxide Synthase 3, Endothelial (NOS3) (Cloud-Clone Corp. Cat. SEA868Hu, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of NOS3 protein was used. The sensitivity was at least 57 μg/ml (0.057 ng/ml), measurement range—from 156 μg/ml to 10000 μg/ml (0.156-10 ng/ml). R-3.0.2 was used for the statistical treatment of the results and data visualization (https://www.r-project.org/). Diagrams resulting from the assay are shown in FIG. 8.

FIG. 8 shows that the transfection of HBdSMc human urinary bladder smooth muscle cells with gene therapy DNA vector VTvafl7-NOS3 results in increased NOS3 protein concentration compared to reference samples, which confirms the ability of the vector to penetrate eukaryotic cells and express NOS3 gene at the protein level. The presented results also confirm the practicability of use of gene therapy DNA vector VTvafl7-NOS3 in order to increase the expression level of NOS3 gene in eukaryotic cells.

### Example 13

Proof of the efficiency and practicability of use of gene therapy DNA vector VTvafl7-VIP carrying the VIP gene in order to increase the expression of VIP protein in mammalian cells.

The change in the VIP protein concentration in conditioned medium from HBdSMC primary human urinary bladder smooth muscle cell culture (ATCC PCS-420-012) was assessed after transfection of these cells with the DNA vector VTvafl7-VIP carrying the human VIP gene.

HBdSMC primary human urinary bladder smooth muscle cell culture was grown in the medium with growth additives prepared using the Vascular Smooth Muscle Cell GroCGRPh Kit (ATCC® PCS-100-042™) under standard conditions (37° C., 5% CO2). To achieve 90% confluence, 24 hours before the transfection procedure, the cells were seeded into a 24-well plate in the quantity of 5×104 cells per well. The 6th generation SuperFect Transfection Reagent (Qiagen, Germany) was used for transfection. The aqueous dendrimer solution without DNA vector (A) and DNA vector VTvafl7 devoid of cDNA of VIP gene (B) were used as a reference, and DNA vector VTvafl7-VIP carrying the human VIP gene was used as the transfected agent. The DNA-dendrimer complex was prepared according to the manufacturer's procedure (QIAGEN, SuperFect Transfection Reagent Handbook, 2002) with some modifications. For cell transfection in one well of a 24-well plate, McCoy's 5A Medium was added to 1p g of DNA vector dissolved in TE buffer to a final volume of 60 μl, then 5 μl of SuperFect Transfection Reagent was added and gently mixed by pipetting five times. The complex was incubated at room temperature for 10-15 minutes. Then the culture medium was taken from the wells, the wells were rinsed with 1 ml of PBS buffer. 350 μl of McCoy's 5A Medium containing 10 μg/ml of gentamicin was added to the resulting complex, mixed gently, and added to the cells. The cells were incubated with the complexes for 2-3 hours at 37° C. in the presence of 5% CO2.

The medium was then removed carefully, and the live cell array was rinsed with 1 ml of PBS buffer. Then, McCoy's 5A Medium containing 10 μg/ml of gentamicin was added and incubated for 24-48 hours at 37° C. in the presence of 5% CO2.

After transfection, 0.1 ml of 1 N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein.

The NOS2 protein was assayed by enzyme-linked immunosorbent assay (ELISA) using the ELISA Kit for Vasoactive Intestinal Peptide (VIP) (Cloud-Clone Corp. Cat. CEA380Hu, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of VIP protein was used. The sensitivity was at least 2.63 μg/ml (0.00263 ng/ml), measurement range—from 6.17 μg/ml to 500 μg/ml (0.00617-0.5 ng/ml). R-3.0.2 was used for the statistical treatment of the results and data visualization (https://www.r-project.org/). Diagrams resulting from the assay are shown in FIG. 9.

FIG. 9 shows that the transfection of HBdSMc human urinary bladder smooth muscle cells with gene therapy DNA vector VTvafl7-VIP results in increased VIP protein concentration compared to reference samples, which confirms the ability of the vector to penetrate eukaryotic cells and express VIP gene at the protein level. The presented results also confirm the practicability of use of gene therapy DNA vector VTvafl7-VIP in order to increase the expression level of VIP gene in eukaryotic cells.

### Example 14

Proof of the efficiency and practicability of use of gene therapy DNA vector VTvafl7-KCNMA1 carrying the KCNMA1 gene in order to increase the expression of KCNMA1 protein in mammalian cells.

The change in the KCNMA1 protein concentration in the cell lysate of HBdSMC primary human urinary bladder smooth muscle cell culture (ATCC PCS-420-012) was assessed after transfection of these cells with the DNA vector VTvafl7-KCNMA1 carrying the human KCNMA1 gene.

HBdSMC primary human urinary bladder smooth muscle cell culture was grown in the medium with growth additives prepared using the Vascular Smooth Muscle Cell GroCGRPh Kit (ATCC® PCS-100-042™) under standard conditions (37° C., 5% CO2). To achieve 90% confluence, 24 hours before the transfection procedure, the cells were seeded into a 24-well plate in the quantity of 5×104 cells per well. The 6th generation SuperFect Transfection Reagent (Qiagen, Germany) was used for transfection. The aqueous dendrimer solution without DNA vector (A) and DNA vector VTvafl7 devoid of cDNA of KCNMA1 gene (B) were used as a reference, and DNA vector VTvafl7-KCNMA1 carrying the human KCNMA1 gene was used as the transfected agent. The DNA-dendrimer complex was prepared according to the manufacturer's procedure (QIAGEN, SuperFect Transfection Reagent Handbook, 2002) with some modifications. For cell transfection in one well of a 24-well plate, McCoy's 5A Medium was added to 1 μg of DNA vector dissolved in TE buffer to a final volume of 60 μl, then 5 μl of SuperFect Transfection Reagent was added and gently mixed by pipetting five times. The complex was incubated at room temperature for 10-15 minutes. Then the culture medium was taken from the wells, the wells were rinsed with 1 ml of PBS buffer. 350 μl of McCoy's 5A Medium containing 10 μg/ml of gentamicin was added to the resulting complex, mixed gently, and added to the cells. The cells were incubated with the complexes for 2-3 hours at 37° C. in the presence of 5% CO2.

The medium was then removed carefully, and the live cell array was rinsed with 1 ml of PBS buffer. Then, McCoy's 5A Medium containing 10 μg/ml of gentamicin was added and incubated for 24-48 hours at 37° C. in the presence of 5% CO2.

After transfection, cells were rinsed three times with PBS, and then 1 ml of PBS was added to the cells and the cells were subjected to freezing/thawing three times. Then the suspension was centrifuged for 15 minutes at 15,000 rpm, and supernatant was collected and used for the quantification and assay of the therapeutic protein.

The KCNMA1 protein was assayed by enzyme-linked immunosorbent assay (ELISA) using the Human KCNMA1/BK ELISA Kit (Sandwich ELISA) (LifeSpan BioScieces Cat. LS-F38925, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of KCNMA1 protein was used.

The sensitivity was at least 0.65 μg/ml (0.00065 ng/ml), measurement range—from 1.23 μg/ml to 100 μg/ml (0.00123-0.1 ng/ml). R-3.0.2 was used for the statistical treatment of the results and data visualization (https://www.r-project.org/). Diagrams resulting from the assay are shown in FIG. 10.

FIG. 10 shows that the transfection of HBdSMc human urinary bladder smooth muscle cells with gene therapy DNA vector VTvafl7-KCNMA1 results in increased KCNMA1 protein concentration compared to reference samples, which confirms the ability of the vector to penetrate eukaryotic cells and express KCNMA1 gene at the protein level. The presented results also confirm the practicability of use of gene therapy DNA vector VTvafl7-KCNMA1 in order to increase the expression level of the KCNMA1 gene in eukaryotic cells.

### Example 15

Proof of the efficiency and practicability of use of gene therapy DNA vector VTvafl7-CGRP carrying the CGRP gene in order to increase the expression of CGRP protein in mammalian cells.

The change in the CGRP protein concentration in the lysate of primary corpus cavernosum penis smooth muscle cell culture was assessed after transfection of these cells with the DNA vector VTvafl7-CGRP carrying the human CGRP gene. Cells were obtained as described in Example 9.

The 6th generation SuperFect Transfection Reagent (Qiagen, Germany) was used for transfection. The aqueous dendrimer solution without DNA vector (A) and DNA vector VTvafl7 devoid of cDNA of CGRP gene (B) were used as a reference, and DNA vector VTvafl7-CGRP carrying the human CGRP gene (C) was used as the transfected agent. Preparation of the DNA dendrimer complex and transfection of primary human corpus cavernosum penis smooth muscle cells were performed according to the procedure described in Example 11.

After transfection, cells were rinsed three times with PBS, and then 1 ml of PBS was added to the cells and the cells were subjected to freezing/thawing three times. Then the suspension was centrifuged for 15 minutes at 15,000 rpm, and supernatant was collected and used for the quantification and assay of the therapeutic protein.

The CGRP protein was assayed by enzyme-linked immunosorbent assay (ELISA) using the ELISA Kit for Calcitonin Gene Related Peptide (CGRP) (Cloud-Clone Corp. Cat. CEA876Hu, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of CGRP protein was used. The sensitivity was at least 5.35 μg/ml (0.00535 ng/ml), measurement range—from 12.35 μg/ml to 1000 μg/ml (0.001235-1 ng/ml). R-3.0.2 was used for the statistical treatment of the results and data visualization (https://www.r-project.org/). Diagrams resulting from the assay are shown in FIG. 11.

FIG. 11 shows that the transfection of primary human corpus cavernosum penis smooth muscle cells with gene therapy DNA vector VTvafl7-CGRP results in increased CGRP protein concentration compared to reference samples, which confirms the ability of the vector to penetrate eukaryotic cells and express the CGRP gene at the protein level. The presented results also confirm the practicability of use of gene therapy DNA vector VTvafl7-CGRP in order to increase the expression level of CGRP gene in eukaryotic cells.

### Example 16

Proof of the efficiency and practicability of use of gene therapy DNA vector VTvafl7-NOS2 carrying the NOS2 gene in order to increase the expression of NOS2 protein in human cells.

To confirm the efficiency of gene therapy DNA vector VTvafl7-NOS2 carrying the therapeutic gene, namely the NOS2 gene, and practicability of its use, changes in NOS2 protein concentration in human skin upon injection of gene therapy DNA vector VTvafl7-NOS2 carrying the human NOS2 gene were assessed.

To analyse changes in the NOS2 protein concentration, gene therapy DNA vector VTvafl7-NOS2 carrying the NOS2 gene was injected into the forearm skin of three patients with concurrent injection of a placebo being gene therapy DNA vector VTvafl7 devoid of cDNA of NOS2 gene.

Patient 1, man, 66 y.o. (P1); Patient 2, woman, 67 y.o. (P2); Patient 3, man, 62 y.o. (P3).

Polyethyleneimine Transfection reagent cGMP grade in-vivo-jetPEI (Polyplus Transfection, France) was used as a transport system. Gene therapy DNA vector VTvafl7-NOS2 containing cDNA of NOS2 gene and gene therapy DNA vector VTvafl7 used as a placebo not containing cDNA of NOS2 gene were dissolved in sterile nuclease-free water. To obtain a gene construct, DNA-cGMP grade in-vivo-jetPEI complexes were prepared according to the manufacturer recommendations.

Gene therapy DNA vector VTvafl7 (placebo) and gene therapy DNA vector VTvafl7-NOS2 carrying the NOS2 gene were injected in the quantity of 1 mg for each genetic construct using the tunnel method with a 30 G needle to the depth of 1.5 mm. The injectate volume of gene therapy DNA vector VTvafl7 (placebo) and gene therapy DNA vector VTvafl7-NOS2 carrying the NOS2 gene was 0.3 ml for each genetic construct. The points of injection of each genetic construct were located at 8 to 10 cm intervals at the forearm site.

The biopsy samples were taken on the 2nd day after the injection of the genetic constructs of gene therapy DNA vectors. The biopsy samples were taken from the patients' skin in the site of injection of gene therapy DNA vector VTvafl7-NOS2 carrying the NOS2 gene (I), gene therapy DNA vector VTvafl7 (placebo) (II), and from intact skin (III) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used in order to assay the therapeutic protein by enzyme-linked immunosorbent assay (ELISA) using ELISA Kit for Nitric Oxide Synthase 2, Inducible (NOS2) (Cloud-Clone Corp. Cat. SEA837Hu, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of NOS2 protein was used. The sensitivity was at least 54 μg/ml (0.057 ng/ml), measurement range—from 156 μg/ml to 10000 μg/ml (0.156-10 ng/ml).

FIG. 12 shows the increased NOS2 protein concentration in the skin of all three patients in the injection site of gene therapy DNA vector VTvafl7-NOS2 carrying the human NOS2 therapeutic gene compared to the NOS2 protein concentration in the injection site of gene therapy DNA vector VTvafl7 (placebo) devoid of the human NOS2 gene, which indicates the efficiency of gene therapy DNA vector VTvafl7-NOS2 and confirms the practicability of its use, in particular upon intracutaneous injection of gene therapy DNA vector in human tissues.

### Example 17

Proof of the efficiency and practicability of use of gene therapy DNA vector VTvafl7-NOS3 carrying the NOS3 gene in order to increase the expression of NOS3 protein in human cells.

To prove the efficiency of gene therapy DNA vector VTvafl7-NOS3 carrying the NOS3 therapeutic gene and practicability of its use, the change in the NOS3 protein concentration in human muscle tissues upon injection of gene therapy DNA vector VTvafl7-NOS3 carrying the therapeutic gene, namely the human NOS3 gene, was assessed.

To analyse changes in the concentration of NOS3 protein, gene therapy DNA vector VTvafl7-NOS3 carrying the NOS3 gene with transport molecule was injected into the skin of three patients with concurrent injection of a placebo being gene therapy DNA vector VTvafl7 devoid of cDNA of NOS3 gene with transport molecule.

Patient 1, woman, 60 y.o. (P1); Patient 2, man, 62 y.o. (P2); Patient 3, man, 63 y.o. (P3). Polyethyleneimine Transfection reagent cGMP grade in-vivo-jetPEI (Polyplus Transfection, France) was used as a transport system; sample preparation was carried out in accordance with the manufacturer's recommendations.

Gene therapy DNA vector VTvafl7 (placebo) and gene therapy DNA vector VTvafl7-NOS3 carrying the NOS3 gene were injected in the quantity of 1 mg for each genetic construct using the tunnel method with a 30 G needle to the depth of around 10 mm. The injectate volume of gene therapy DNA vector VTvafl7 (placebo) and gene therapy DNA vector VTvafl7-NOS3 carrying the NOS3 gene was 0.3 ml for each genetic construct. The points of injection of each genetic construct were located medially at 8 to 10 cm intervals.

The biopsy samples were taken on the 2nd day after the injection of the genetic constructs of gene therapy DNA vectors. The biopsy samples were taken from the patients' muscle tissues in the site of injection of gene therapy DNA vector VTvafl7-NOS3 carrying the NOS3 gene (I), gene therapy DNA vector VTvafl7 (placebo) (II), and intact site of gastrocnemius muscle (III) using the skin biopsy device MAGNUM (BARD, USA). The skin of patients in the biopsy site was preliminarily rinsed with sterile saline and anaesthetised with a lidocaine solution. The biopsy sample size was ca. 20 mm3, and the weight was up to 22 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein.

The NOS3 protein was assayed by enzyme-linked immunosorbent assay (ELISA) as described in Example 12 with optical density detection using ChemWell Automated EIA and Chemistry Analyser (Awareness Technology Inc., USA).

Diagrams resulting from the assay are shown in FIG. 13.

FIG. 13 shows the increased NOS3 protein concentration in the gastrocnemius muscle of all three patients in the injection site of gene therapy DNA vector VTvafl7-NOS3 carrying the therapeutic gene, namely NOS3 gene, compared to the NOS3 protein concentration in the injection site of gene therapy DNA vector VTvafl7 (placebo) devoid of the human NOS3 gene, which indicates the efficiency of gene therapy DNA vector VTvafl7-NOS3 and confirms the practicability of its use, in particular upon intramuscular injection of gene therapy DNA vector in human tissues.

### Example 18

Proof of the efficiency and practicability of use of gene therapy DNA vector VTvafl7-KCNMA1 carrying the KCNMA1 gene in order to increase the expression level of KCNMAI protein in mammalian tissues.

The change in the KCNMA1 protein concentration in the corpus cavernosum penis biopsy was assessed upon intracavernous penile injection of gene therapy DNA vector VTvafl7-KCNMA1.

Solution for injection that constitutes a mixture of DNA vector with a transport system based on polyethyleneimine Transfection reagent cGMP grade in-vivo-jetPEI (Polyplus Transfection, France) was prepared according to the manufacturer's instructions. The injectate volume was 0.75 ml with a DNA concentration of 1 μg/μl. Gene therapy DNA vector VTvafl7-KCNMA1 carrying the KCNMA1 gene was intracavernously injected into the right corpus cavernosum penis by three injections with points of injection located proximally 1 cm apart from each other using a 30 G needle. Gene therapy DNA vector VTvafl7 (placebo) was intracavernously injected into the left corpus cavernosum penis by three injections with points of injection located proximally 1 cm apart from each other with a 30 G needle. After injection of gene therapy DNA vector VTvafl7-KCNMA1 and placebo in order to ensure the maximum possible time for the plasmid to remain in the injection area, a compression tourniquet was applied to the base of the penis for 20 minutes.

The biopsy samples were taken on the 2nd day after the injection of the gene therapy DNA vectors. The biopsy samples were taken from the patients' corpus cavernosum penis in the site of injection of gene therapy DNA vector VTvafl7-KCNMA1 carrying the KCNMA1 gene (I), gene therapy DNA vector VTvafl7 (placebo)(II), and intact site of corpus cavernosum penis (III) using the skin biopsy device MAGNUM (BARD, USA). The patient skin in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein as described in Example 14.

Diagrams resulting from the assay are shown in FIG. 14.

FIG. 14 shows that in the human corpus cavernosum penis biopsy from the site of injection of gene therapy DNA vector VTvafl7-KCNMA1 carrying the KCNMA1 gene, an increase in the KCNMA1 protein concentration is observed compared to the control group II (placebo) and the control group III (intact site). The obtained results show the efficiency of intracavernous injection of gene therapy DNA vector and practicability of use for the upregulation of the expression level of therapeutic protein in mammalian tissues.

### Example 19

Proof of the efficiency of gene therapy DNA vector VTvafl7-NOS2 carrying the NOS2 gene and practicability of its use in order to increase the expression level of the NOS2 protein in human tissues by injecting autologous fibroblasts transfected with gene therapy DNA vector VTvafl7-NOS2.

To confirm the efficiency of gene therapy DNA vector VTvafl7-NOS2 carrying the NOS2 gene and practicability of its use, changes in the NOS2 protein concentration in patient's skin upon injection of autologous fibroblast culture of the same patient transfected with gene therapy DNA vector VTvafl7-NOS2 were assessed.

The appropriate autologous fibroblast culture transfected with the gene therapy DNA vector VTvafl7-NOS2 carrying the NOS2 gene was injected into the patient's forearm skin with concurrent injection of a placebo in the form of autologous fibroblast culture transfected with gene therapy DNA vector VTvafl7 not carrying the NOS2 gene.

The human primary fibroblast culture was isolated from the patient skin biopsy specimens. Biopsy specimens of the skin from the area protected by ultraviolet, namely behind the ear or on the inner lateral side of the elbow, were taken using the skin biopsy device Epitheasy 3.5 (Medax SRL, Italy). The biopsy sample was ca. 10 mm and ca. 11 mg. The patient's skin was preliminarily rinsed with sterile saline and anaesthetised with a lidocaine solution. The primary cell culture was cultivated at 37° C. in the presence of 5% CO2, in the DMEM medium with 10% fetal bovine serum and 100 U/ml of ampicillin. The passage and change of culture medium were performed every 2 days. Total duration of culture growth did not exceed 25-30 days. Then an aliquot of 5×104 cells was taken from the cell culture. The patient's fibroblast culture was transfected with the gene therapy DNA vector VTvafl7-NOS2 carrying the NOS2 gene or placebo, i.e. VTvafl7 vector not carrying the NOS2 therapeutic gene.

The transfection was carried out using a cationic polymer such as polyethyleneimine JETPEI (Polyplus transfection, France), according to the manufacturer's instructions. The cells were cultured for 72 hours and then injected into the patient. Injection of autologous fibroblast culture of the patient transfected with gene therapy DNA vector VTvafl7-NOS2 and autologous fibroblast culture of the patient transfected with gene therapy DNA vector VTvafl7 as a placebo was performed in the forearm using the tunnel method with a 13 mm long 30 G needle to the depth of approximately 3 mm. The concentration of the modified autologous fibroblasts in the injected suspension was approximately 5 mln cells per 1 ml of the suspension, the dose of the injected cells did not exceed 15 mln. The points of injection of the autologous fibroblast culture were located at 8 to 10 cm intervals.

Biopsy samples were taken on the 4th day after the injection of autologous fibroblast culture transfected with the gene therapy DNA vector VTvafl7-NOS2 carrying the therapeutic gene, namely NOS2 gene, and placebo. Biopsy was taken from the patient's skin in the site of injection of autologous fibroblast culture transfected with gene therapy DNA vector VTvafl7-NOS2 carrying the therapeutic gene, namely NOS2 gene (C), autologous fibroblast culture transfected with gene therapy DNA vector VTvafl7 not carrying the NOS2 therapeutic gene (placebo) (B), as well as from intact skin site (A) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein as described in Example 11.

Diagrams resulting from the assay are shown in FIG. 15.

FIG. 15 shows the increased concentration of NOS2 protein in the area of the patient's skin in the injection site of autologous fibroblast culture transfected with the gene therapy DNA vector VTvafl7-NOS2 carrying the NOS2 gene compared to the NOS2 protein concentration in the injection site of autologous fibroblast culture transfected with the gene therapy DNA vector VTvafl7 that does not carry the NOS2 gene (placebo), which indicates the efficiency of gene therapy DNA vector VTvafl7-NOS2 and practicability of its use in order to increase the expression level of NOS2 in human tissues, in particular upon injection of autologous fibroblasts transfected with the gene therapy DNA vector VTvafl7-NOS2 into the skin.

### Example 20

Proof of the efficiency and practicability of use of gene therapy DNA vector VTvafl7-KCNMA1 carrying the KCNMA1 gene in order to increase the expression of KCNMA1 protein in mammalian cells.

To confirm the efficiency of gene therapy DNA vector VTvafl7-KCNMA1 carrying the KCNMA1 gene, the change in mRNA accumulation of KCNMA1 therapeutic gene in BAOSMC bovine aortic smooth muscle cells (Genlantis) 48 hours after their transfection with gene therapy DNA vector VTvafl7-KCNMA1 carrying the human KCNMA1 gene were assessed compared to BEND reference cells transfected with gene therapy DNA vector VTvafl7 not carrying the human KCNMA1 gene (placebo).

BAOSMC bovine aortic smooth muscle cell culture (Genlantis) was grown in Bovine Smooth Muscle Cell Growth Medium (Sigma B311F-500) with the addition of bovine serum up to 10% (Paneco, Russia). Transfection with gene therapy DNA vector VTvafl7-KCNMAI carrying the human KCNMA1 gene and DNA vector VTvafl7, RNA extraction, reverse transcription reaction, PCR amplification, and data analysis were performed as described in Example 9. Bull/cow actin gene (ACT) listed in the GenBank database under number AH001130.2 was used as a reference gene.

Diagrams resulting from the assay are shown in FIG. 16.

FIG. 16 shows that cDNA amplicons were accumulated faster than in reference samples of cells as a result of transfection of BAOSMC bovine aortic smooth muscle cells with gene therapy DNA vector VTvafl7-KCNMA1, which indicates higher mRNA level of human KCNMA1 gene in the cells after transfection with gene therapy DNA vector VTvafl7-KCNMA1. The presented results confirm the practicability of use of gene therapy DNA vector VTvafl7-KCNMA1 in order to increase the expression level of KCNMA1 gene in mammalian cells.

### Example 21

Escherichia coli strain SCS110-AF/VTvafl7-NOS2, or Escherichia coli strain SCS110-AF/VTvafl7-NOS3, or Escherichia coli strain SCS110-AF/VTvafl7-VIP, or Escherichia coli strain SCS110-AF/VTvafl7-KCNMA1, or Escherichia coli strain SCS110-AF/VTvafl7-CGRP carrying gene therapy DNA vector, method of production thereof.

The construction of strain for the production of gene therapy DNA vector based on gene therapy DNA vector VTvafl7 carrying the therapeutic gene on an industrial scale selected from the group of the following genes: NOS2, NOS3, VIP, KCNMA1, and CGRP, namely Escherichia coli strain SCS110-AF/VTvafl7-NOS2, or Escherichia coli strain SCS110-AF/VTvafl7-NOS3, or Escherichia coli strain SCS110-AF/VTvafl7-VIP, or Escherichia coli strain SCS110-AF/VTvafl7-KCNMA1, or Escherichia coli strain SCS110-AF/VTvafl7-CGRP carrying the gene therapy DNA vector VTvafl7-NOS2, or VTvafl7-NOS3, or VTvafl7-VIP, or VTvafl7-KCNMA1, or VTvafl7-CGRP, respectively, for its production allowing for antibiotic-free selection involves making electrocompetent cells of Escherichia coli strain SCS1O-AF and subjecting these cells to electroporation with gene therapy DNA vector VTvafl7-NOS2, or DNA vector VTvafl7-NOS3, or DNA vector VTvafl7-VIP, or DNA vector VTvafl7-KCNMA1, or DNA vector VTvafl7-CGRP. After that, the cells were poured into agar plates (Petri dishes) with a selective medium containing yeastrel, peptone, 6% sucrose, and 10 μg/ml of chloramphenicol. At the same time, production of Escherichia coli strain SCS110-AF for the production of gene therapy DNA vector VTvafl7 or gene therapy DNA vectors based on it allowing for antibiotic-free positive selection involves constructing a 64 bp linear DNA fragment that contains regulatory element RNA-IN of transposon Tn10 allowing for antibiotic-free positive selection, a 1422 bp levansucrase gene sacB, the product of which ensures selection within a sucrose-containing medium, a 763 bp chloramphenicol resistance gene catR required for the selection of strain clones in which homologous recombination occurs, and two homologous sequences, 329 bp and 233 bp, ensuring homologous recombination in the region of gene recA concurrent with gene inactivation, and then the Escherichia coli cells are transformed by electroporation, and clones surviving in a medium containing 10 μg/ml of chloramphenicol are selected. The obtained strains for the production were included in the collection of the National Biological Resource Centre—Russian National Collection of Industrial Microorganisms (NBRC RNCIM), RF and NCIMB Patent Deposit Service, UK under the following registration numbers: Escherichia coli strain SCS110-AF/VTvafl7-NOS2—registered at the Russian National Collection of Industrial Microorganisms under number B-13323, date of deposit 12.12.2018, INTERNATIONAL DEPOSITARY AUTHORITY No. NCIMB 43244, date of deposit 08.11.2018, Escherichia coli strain SCS110-AF/VTvafl7-NOS3—registered at the Russian National Collection of Industrial Microorganisms under number B-13255, date of deposit 24.09.2018, INTERNATIONAL DEPOSITARY AUTHORITY No. NCIMB 43206,date of deposit 20.09.2018, Escherichia coli strain Escherichia coli SCS110-AF/VTvafl7-VIP—registered at the Russian National Collection of Industrial Microorganisms under number B-13252, date of deposit 24.09.2018, INTERNATIONAL DEPOSITARY AUTHORITY No. NCIMB 43204, date of deposit 20.09.2018, Escherichia coli strain SCS110-AF/VTvafl7-KCNMA1—registered at the Russian National Collection of Industrial Microorganisms under number B-13257, date of deposit 24.09.2018, INTERNATIONAL DEPOSITARY AUTHORITY No. NCIMB 43205, date of deposit 20.09.2018, Escherichia coli strain SCS110-AF/VTvafl7-CGRP—registered at the Russian National Collection of Industrial Microorganisms under number B-13277, date of deposit 16.10.2018, INTERNATIONAL DEPOSITARY AUTHORITY No. NCIMB 43306, date of deposit 13.12.2018.

### Example 22

The method for scaling up of the gene therapy DNA vector based on gene therapy DNA vector VTvafl7 carrying the therapeutic gene selected from the group of NOS2, NOS3, VIP, KCNMA1, and CGRP to an industrial scale.

To confirm the producibility and constructability of gene therapy DNA vector VTvafl7-NOS2 (SEQ ID No. 1), or VTvafl7-NOS3 (SEQ ID No. 2), or VTvafl7-VIP (SEQ ID No. 3), or VTvafl7-KCNMA1 (SEQ ID No. 4), or VTvafl7-CGRP (SEQ ID No. 5) on an industrial scale, large-scale fermentation of Escherichia coli strain SCS110-AF/VTvafl7-NOS2, or Escherichia coli strain SCS110-AF/VTvafl7-NOS3, or Escherichia coli strain SCS110-AF/VTvafl7-VIP, or Escherichia coli strain SCS110-AF/VTvafl7-KCNMA1, or Escherichia coli strain SCS110-AF/VTvafl7-CGRP each containing gene therapy DNA vector VTvafl7 carrying the therapeutic gene, namely NOS2, or NOS3, or VIP, or KCNMA1, or CGRP was performed. Each Escherichia coli strain SCS110-AF/VTvafl7-NOS2, or Escherichia coli strain SCS110-AF/VTvafl7-NOS3, or Escherichia coli strain SCS110-AF/VTvafl7-VIP, or Escherichia coli strain SCS110-AF/VTvafl7-KCNMA1, or Escherichia coli strain SCS110-AF/VTvafl7-CGRP was produced on the basis of Escherichia coli strain SCS110-AF (Cell and Gene Therapy LLC, United Kingdom) as described in Example 21 by electroporation of competent cells of this strain with the gene therapy DNA vector VTvafl7-NOS2, or VTvafl7-NOS3, or VTvafl7-VIP, or VTvafl7-KCNMA1, or VTvafl7-CGRP carrying the therapeutic gene, namely NOS2, or NOS3, or VIP, or KCNMA1, or CGRP, with further inoculation of transformed cells into agar plates (Petri dishes) with a selective medium containing yeastrel, peptone, and 6% sucrose, and selection of individual clones.

Fermentation of Escherichia coli SCS110-AFNTvafl7-NOS2 carrying gene therapy DNA vector VTvafl7-NOS2 was performed in a 101 fermenter with subsequent extraction of gene therapy DNA vector VTvafl7-NOS2.

For the fermentation of Escherichia coli strain SCS110-AF/VTvafl7-NOS2, a medium was prepared containing (per 101 of volume): 100 g of tryptone and 50 g of yeastrel (Becton Dickinson, USA); then the medium was diluted with water to 8800 ml and autoclaved at 121° C. for 20 minutes, and then 1200 ml of 50% (w/v) sucrose was added. After that, the seed culture of Escherichia coli strain SCS110-AF/VTvafl7-NOS2 was inoculated into a culture flask in the volume of 100 ml. The culture was incubated in an incubator shaker for 16 hours at 30° C. The seed culture was transferred to the Techfors S bioreactor (Infors HT, Switzerland) and grown to a stationary phase. The process was controlled by measuring optical density of the culture at 600 nm. The cells were pelleted for 30 minutes at 5,000-10,000 g. Supernatant was removed, and the cell pellet was re-suspended in 10% (by volume) phosphate buffered saline. The cells were centrifuged again for 30 minutes at 5,000-10,000 g. Supernatant was removed, a solution of 20 mM TrisCl, 1 mM EDTA, 200 g/l sucrose, pH 8.0 was added to the cell pellet in the volume of 1000 ml, and the mixture was stirred thoroughly to a homogenised suspension. Then egg lysozyme solution was added to the final concentration of 100 μg/ml. The mixture was incubated for 20 minutes on ice while stirring gently. Then 2500 ml of 0.2M NaOH, 10 g/l sodium dodecyl sulphate (SDS) was added, the mixture was incubated for 10 minutes on ice while stirring gently, then 3500 ml of 3M sodium acetate, 2M acetic acid, pH 5-5.5 was added, and the mixture was incubated for 10 minutes on ice while stirring gently. The resulting sample was centrifuged for 20-30 minutes at 15,000 g or a greater value. The solution was decanted delicately, and residual precipitate was removed by passing through a coarse filter (filter paper). Then, RNase A (Sigma, USA) was added to the final concentration of 20 μg/ml, and the solution was incubated overnight for 16 hours at room temperature. The solution was then centrifuged for 20-30 minutes at 15,000 g and passed through a 0.45 μm membrane filter (Millipore, USA). Then, ultrafiltration was performed with a 100 kDa membrane (Millipore, USA) and the mixture was diluted to the initial volume with a buffer solution of 25 mM TrisCl, pH 7.0. This manipulation was performed three to four times. The solution was applied to the column with 250 ml of DEAE Sepharose HP (GE, USA), equilibrated with 25 mM TrisCl, pH 7.0. After the application of the sample, the column was washed with three volumes of the same solution, and then gene therapy DNA vector VTvafl7-NOS2 was eluted using a linear gradient of 25 mM Tris-HCl, pH 7.0, to obtain a solution of 25 mM Tris-HCl, pH 7.0, 1M NaCl, five times the volume of the column. The elution process was controlled by measuring optical density of the run-off solution at 260 nm. Chromatographic fractions containing gene therapy DNA vector VTvafl7-NOS2 were joined together and subjected to gel filtration using Superdex 200 (GE, USA). The column was equilibrated with phosphate buffered saline. The elution process was controlled by measuring optical density of the run-off solution at 260 nm, and the fractions were analysed by agarose gel electrophoresis. The fractions containing gene therapy DNA vector VTvafl7-NOS2 were joined together and stored at −20° C. To assess the process reproducibility, the indicated processing operations were repeated five times. All processing operations for Escherichia coli strain SCS110-AF/VTvafl7-NOS3, or Escherichia coli strain SCS110-AF/VTvafl7-VIP, or Escherichia coli strain SCS110-AF/VTvafl7-KCNMA1, or Escherichia coli strain SCS110-AF/VTvafl7-CGRP were performed in a similar way.

The process reproducibility and quantitative characteristics of final product yield confirm the producibility and constructability of gene therapy DNA vector VTvafl7-NOS2, or VTvafl7-NOS3, or VTvafl7-VIP, or VTvafl7-KCNMA1, or VTvafl7-CGRP on an industrial scale.

Thus, the produced gene therapy DNA vector containing the therapeutic gene can be used to deliver it to the cells of human beings and animals that experience reduced or insufficient expression of protein encoded by this gene, thus ensuring the desired therapeutic effect.

The purpose set in this invention, namely the construction of the gene therapy DNA vectors in order to increase the expression NOS2, NOS3, VIP, KCNMA1, and CGRP genes that combine the following properties:
I) Efficiency of the upregulation of expression of therapeutic genes in eukaryotic cells,

Possibility of safe use in gene therapy of human beings and animals due to the absence of regulatory elements representing the nucleotide sequences of viral genomes in the gene therapy DNA vector,

Possibility of safe use in the gene therapy of human beings and animals due to the absence of antibiotic resistance genes in the gene therapy DNA vector,
II) Producibility and constructability in the strains on an industrial scale,
III) as well as the purpose of the construction of strains carrying these gene therapy DNA vectors for the production of these gene therapy DNA vectors has been achieved, which is supported by the following examples:
for Item I—Example 1, 2, 3, 4, 5; 6; 7; 8; 9; 10; 11; 12; 13; 14; 15; 16; 17; 18; 19; 20.
for Item II—Example 1, 2, 3, 4, 5;
for Item III—Example 1, 2, 3, 4, 5;
for Item IV—Example 21, 22.

### INDUSTRIAL APPLICABILITY

All the examples listed above confirm the industrial applicability of the proposed gene therapy DNA vector based on gene therapy DNA vector VTvafl7 carrying the therapeutic gene selected from the group of NOS2, NOS3, VIP, KCNMA1, and CGRP genes in order to increase the expression level of these therapeutic genes, Escherichia coli strain SCS110-AF/VTvafl7-NOS2, or Escherichia coli strain SCS110-AF/VTvafl7-NOS3, or Escherichia coli strain SCS110-AF/VTvafl7-VIP, or Escherichia coli strain SCS110-AF/VTvafl7-KCNMA1, or Escherichia coli strain SCS110-AF/VTvaf17-CGRP carrying gene therapy DNA vector, and method of its production on an industrial scale.

### LIST OF ABBREVIATIONS

VTvafl7—Gene therapy vector devoid of sequences of viral genomes and antibiotic resistance markers (vector therapeutic virus-antibiotic-free)
DNA—Deoxyribonucleic acid
cDNA—Complementary deoxyribonucleic acid
RNA—Ribonucleic acid
mRNA—Messenger ribonucleic acid
bp—base pair
PCR—Polymerase chain reaction
ml—millilitre, μl—microlitre
mm3—cubic millimetre
1—litre
μg—microgram
mg—milligram
g—gram
μM—micromol
mM—millimol
min—minute
s—second
rpm—rotations per minute
nm—nanometre
cm—centimetre
mW—milliwatt
RFU—Relative fluorescence unit
PBS—Phosphate buffered saline

### LIST OF REFERENCES

1. Molecular Biology, 2011, Vol. 45, No. 1, p. 44-55
2. Application No. WO1994016718A1
3. Application No. US20040120930A1
4. Patent No. U.S. Pat. No. 5,594,032A
5. Patent No. U.S. Pat. No. 8,536,146B2
6. Patent No. U.S. Pat. No. 9,550,998B2
7. Barbato JE, Tzeng E. iNOS gene transfer for graft disease. Trends Cardiovasc Med. 2004 Oct;14(7):267-72.
8. Bivalacqua T J, Champion H C, Abdel-Mageedgfntyn A B, Kadowitz P J, Hellstrom W J. Gene
9. Bivalacqua T J, Champion H C, Mehta Y S, Abdel-Mageed A B, Sikka S C, Ignarro L J, Kadowitz P J, Hellstrom W J. Adenoviral gene transfer of endothelial nitric oxide synthase (eNOS) to the penis improves age-related erectile dysfunction in the rat. Int J Impot Res. 2000 Sep;12 Suppl 3: S8-17.
10. Chen H, Yang H, Yue H, Strappe P M, Xia P, Pan L, Zhang Y, Chai S, Chen S, Ma L, Wang L. Mesenchymal Stem Cells Expressing eNOS and a Cavl Mutant Inhibit Vascular Smooth Muscle Cell Proliferation in a Rat Model of Pulmonary Hypertension. Heart Lung Circ. 2017 May; 26(5):509-518.
11. Chevalier R L. Promise for gene therapy in obstructive nephropathy. Kidney Int. 2004 Oct;66(4):1709-10.
12. Cobb J.P., R.S. Hotchkiss, P.E. Swanson, K. Chang, Y. Qiu, V.E. Laubach, I.E. Karl, T.G. Buchman, Inducible nitric oxide synthase (iNOS) gene deficiency increases themortality of sepsis in mice, Surgery 126 (1999) 438-442.
13. Delgado M, Abad C, Martinez C, Leceta J, Gomariz R P. Vasoactive intestinal peptide prevents experimental arthritis by downregulating both autoimmune and inflammatory components of the disease. Nat Med. 2001 May; 7(5):563-8.
14. Draft Guideline on the quality, non-clinical and clinical aspects of gene therapy medicinal products, http://www.ema.europa.eu/docs/en_GB/document_library/Scientific_guideline/2015/05/WC50 0187020.pdf
15. Dran G I, Fernandez G C, Rubel C J, Bermejo E, Gomez S, Meiss R, Isturiz M A,Palermo M S. Protective role of nitric oxide in mice with Shiga toxin-induced hemolytic uremic syndrome. Kidney Int. 2002 October;62(4):1338-48.
16. El Karim I A, Linden G J, Orr D F, Lundy F T. Antimicrobial activity of neuropeptides against a range of micro-organisms from skin, oral, respiratory and gastrointestinal tract sites. J Neuroimmunol. 2008 Aug. 30; 200(1-2):11-6.
17. Fang Z, Yang Q, Xiong W, Li G H, Liao H, Xiao J, Li F. Effect of CGRP-adenoviral vector transduction on the osteoblastic differentiation of rat adipose-derived stem cells. PLoS One. 2013 Aug. 30; 8(8):e72738.
18. Granath A, Norrby-Teglund A, Uddman R, Cardell L O. Reduced iNOS expression in adenoids from children with otitis media with effusion. Pediatr Allergy Immunol. 2010 Dec;21(8):1151-6. doi: 10.1111fj.1399-3038.2010.01020.x.
19. Guan C X, Zhang M, Qin X Q, Cui Y R, Luo Z Q, Bai H B, Fang X. Vasoactive intestinal peptide enhances wound healing and proliferation of human bronchial epithelial cells. Peptides. 2006 Dec;27(12):3107-14. Epub 2006 Sep 11.
20. Guideline on the quality, non-clinical and clinical aspects of gene therapy medicinal Products EMA/CAT/80183/2014
21. Hamidi S A, Lin R Z, Szema A M, Lyubsky S, Jiang Y P, Said S I. VIP and endothelin receptor antagonist: an effective combination against experimental pulmonary arterial hypertension. Respir Res. 2011 Oct. 26; 12:141.
22. He Y, He W, Qin G, Luo J, Xiao M. Transplantation KCNMA1 modified bone marrow-mesenchymal stem cell therapy for diabetes mellitus-induced erectile dysfunction. Andrologia. 2014 June;46(5):479-86.
23. Hornstein B D, Roman D, Arevalo-Soliz L M, Engevik M A, Zechiedrich L. Effects of Circular DNA Length on Transfection Efficiency by Electroporation into HeLa Cells. Ceña V, ed. PLoS ONE. 2016;11(12):e0167537. doi:10.1371/journal.pone.0167537.
24. Igamonti E, Touvier T, Clementi E, Manfredi A A, Brunelli S, Rovere-Querini P. Requirement of inducible nitric oxide synthase for skeletal muscle regeneration after acute damage. J Immunol. 2013 Feb. 15; 190(4):1767-77.
25. Jiang B, Du L, Flynn R, Dronadula N, Zhang J, Kim F, Dichek D. Overexpression of endothelial nitric oxide synthase improves endothelium-dependent vasodilation in arteries infused with helper-dependent adenovirus. Hum Gene Ther. 2012 November; 23(11):1166-75.
26. Kato I, Suzuki Y, Akabane A, Yonekura H, Tanaka 0, Kondo H, Takasawa S, Yoshimoto T, Okamoto H. Enhancement of glucose-induced insulin secretion in transgenic mice overexpressing human VIP gene in pancreatic beta cells. Ann N Y Acad Sci. 1996 Dec. 26; 805:232-42.
27. Li L, Petrovsky N. Molecular mechanisms for enhanced DNA vaccine immunogenicity. Expert Rev Vaccines. 2016; 15(3):313-29
28. Ma G, Liu H, Hua Q, Wang M, Du M, Lin Y, Ge Y, Gong W, Zhao Q, Qiang F, Tao G,Zhang Z, Chu H. KCNMA1 cooperating with PTK2 is a novel tumour suppressor in gastric cancer and is associated with disease outcome. Mol Cancer. 2017 Feb. 23; 16(1):46.
29. Mairhofer J, Grabherr R. Rational vector design for efficient non-viral gene delivery: challenges facing the use of plasmid DNA. Mol Biotechnol. 2008.39(2):97-104
30. Manzanares D, Srinivasan M, Salathe S T, Ivonnet P, Baumlin N, Dennis J S, Conner G E, Salathe M. IFN-7-mediated reduction of large-conductance, Ca2+-activated, voltage-dependent K+(B K) channel activity in airway epithelial cells leads to mucociliary dysfunction. Am J Physiol Lung Cell Mol Physiol. 2014 Mar. 1; 306(5):L453-62.
31. Marquez-Rodas I, Longo F, Rothlin R P, Balfagon G. Pathophysiology and therapeutic possibilities of calcitonin gene-related peptide in hypertension. J Physiol Biochem. 2006 Mar;62(1):45-56.
32. Melman A, Bar-Chama N, McCullough A, Davies K, Christ G. hMaxi-K gene transfer in males with erectile dysfunction: results of the first human trial. Hum Gene Ther. 2006 Dec;17(12):1165-76. PubMed PMID: 17134370.
33. Reflection paper on design modifications of gene therapy medicinal products during development/14 Dec. 2011 EMA/CAT/GTWP/44236/2009 Committee for advanced therapies
34. Schwentker A, Billiar T R.//World J Surg. 2002 July;26(7):772-8. Chevalier R L.//Kidney Int. 2004 October;66(4):1709-10. Igamonti E et al.//J Immunol. 2013 Feb. 15; 190(4):1767-77.
35. Schwentker A, Billiar T R. Inducible nitric oxide synthase: from cloning to therapeutic applications. World J Surg. 2002 July;26(7):772-8.
36. Shah G, Zhang G, Chen F, Cao Y, Kalyanaraman B, See W A. iNOS expression and N O production contribute to the direct effects of BCG on urothelial carcinoma cell biology. Urol Oncol. 2014 January;32(1):45.el-9.
37. She F, Sun W, Mao J M, Wang X. Calcitonin gene-related peptide gene therapy suppresses reactive oxygen species in the pancreas and prevents mice from autoimmune diabetes. Sheng Li Xue Bao. 2003 Dec. 25; 55(6):625-32.
38. Tabarki B, AlMajhad N, AiHashem A, Shaheen R, Alkuraya F S. Homozygous KCNMA1 mutation as a cause of cerebellar atrophy, developmental delay and seizures. Hum Genet. 2016 November; 135(11):1295-1298.
39. Tao J, Zhou Z L, Wu B, Shi J, Chen X M, Ji Y H. Recombinant expression and functional characterization of martentoxin: a selective inhibitor for B K channel (α+β4). Toxins (Basel). 2014 Apr. 22; 6(4):1419-33.
40. Tasyurek H M, Eksi Y E, Sanlioglu A D, Altunbas H A, Balci M K, Griffith T S, Sanlioglu S. HIV-based lentivirus-mediated vasoactive intestinal peptide gene delivery protects against DIO animal model of Type 2 diabetes. Gene Ther. 2018 July;25(4):269-283.
41. transfer of prepro-calcitonin gene-related peptide restores erectile function in the aged rat. Biol Reprod. 2001 November; 65(5):1371-7.
42. Tsuji S, Iharada A, Kimata T, Shimo T, Hirabayashi M, Kaneko K. Production of nitric oxide is lower in Shiga toxin-stimulated neutrophils of infants compared to those of children or adults. Tohoku J Exp Med. 2012 November; 228(3):247-52.
43. Vacas E, Fernindez-Martinez A B, Bajo A M, Sanchez-Chapado M, Schally A V, Prieto J C, Carmena M J. Vasoactive intestinal peptide (VIP) inhibits human renal cell carcinoma proliferation. Biochim Biophys Acta. 2012 October;1823(10):1676-85.
44. Wang T, Li M, Yuan H, Zhan Y, Xu H, Wang S, Yang W, Liu J, Ye Z, Li L C. saRNA guided iNOS up-regulation improves erectile function of diabetic rats. J Urol. 2013 Aug;190(2):790-8.
45. Wang W, Sun W, Wang X. Intramuscular gene transfer of CGRP inhibits neointimal hyperplasia after balloon injury in the rat abdominal aorta. Am J Physiol HeartCirc Physiol. 2004 Oct;287(4):H1582-9.
46. Wei L, Zhu W, Xia L, Yang Y, Liu H, Shen J, Zhu J, Xu Y, Yang Z, Wang C. Therapeutic effect of eNOS-transfected endothelial progenitor cells on hemodynamic pulmonary arterial hypertension. Hypertens Res. 2013 May; 36(5):414-21.
47. Wyllie M G. The anatomy of drug development: Invicorp, a product before its time. BJU Int. 2010 September;106(5):723-4.
48. Xiang L, Ma L, Wei N, Wang T, Yao Q, Yang B, Xiong Y, Wu Y, Gong P. Effect of lentiviral vector overexpression a-calcitonin gene-related peptide on titanium implant osseointegration in a-CGRP-deficient mice. Bone. 2017 January;94:135-140.
49. Zhao C X, Xu X, Cui Y, Wang P, Wei X, Yang S, Edin M L, Zeldin D C, Wang D W. Increased endothelial nitric-oxide synthase expression reduces hypertension and hyperinsulinemia in fructose-treated rats. J Pharmacol Exp Ther. 2009 February;328(2):610-20.

1. Gene therapy DNA vector based on gene therapy DNA vector VTvafl7 for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction, while the gene therapy DNA vector has the coding region of NOS2 therapeutic gene cloned to gene therapy DNA vector VTvafl7 resulting in gene therapy DNA vector VTvafl7-NOS2 that has nucleotide sequence SEQ ID No. 1.

2. Gene therapy DNA vector based on gene therapy DNA vector VTvafl7 for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction, while the gene therapy DNA vector has the coding region of NOS3 therapeutic gene cloned to gene therapy DNA vector VTvafl7 resulting in gene therapy DNA vector VTvafl7-NOS3 that has nucleotide sequence SEQ ID No. 2.

3. Gene therapy DNA vector based on gene therapy DNA vector VTvafl7 for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction, while the gene therapy DNA vector has the coding region of VIP therapeutic gene cloned to gene therapy DNA vector VTvafl7 resulting in gene therapy DNA vector VTvafl7-VIP that has nucleotide sequence SEQ ID No. 3.

4. Gene therapy DNA vector based on gene therapy DNA vector VTvafl7 for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction, while the gene therapy DNA vector has the coding region of KCNMAI therapeutic gene cloned to gene therapy DNA vector VTvafl7 resulting in gene therapy DNA vector VTvafl7-KCNMA1 that has nucleotide sequence SEQ ID No. 4.

5. Gene therapy DNA vector based on gene therapy DNA vector VTvafl7 for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction, while the gene therapy DNA vector has the coding region of CGRP therapeutic gene cloned to gene therapy DNA vector VTvafl7 resulting in gene therapy DNA vector VTvafl7-CGRP that has nucleotide sequence SEQ ID No. 5.

6. Gene therapy DNA vector based on gene therapy DNA vector VTvafl7 carrying NOS2, NOS3, VIP, KCNMA1, or CGRP therapeutic gene as per claim 1, 2, 3, 4, or 5. Said gene therapy DNA vectors are unique due to the fact that each of the constructed gene therapy DNA vectors: VTvafl7-NOS2, or VTvafl7-NOS3, or VTvafl7-VIP, or VTvafl7-KCNMA1, or VTvafl7-CGRP as per claim 1, 2, 3, 4, or 5 due to the limited size of VTvafl7 vector part not exceeding 3200 bp has the ability to efficiently penetrate into human and animal cells and express the NOS2, or NOS3, or VIP, or KCNMA1, or CGRP therapeutic gene cloned to it.

7. Gene therapy DNA vector based on gene therapy DNA vector VTvafl7 carrying NOS2, NOS3, VIP, KCNMA1, or CGRP therapeutic gene as per claim 1, 2, 3, 4, or 5. Said gene therapy DNA vectors are unique due to the fact that each of the constructed gene therapy DNA vectors: VTvafl7-NOS2, or VTvafl7-NOS3, or VTvafl7-VIP, or VTvafl7-KCNMA1, or VTvafl7-CGRP as per claim 1, 2, 3, 4, or 5 uses nucleotide sequences that are not antibiotic resistance genes, virus genes, or regulatory elements of viral genomes as structure elements, which ensures its safe use for gene therapy in humans and animals.

8. A method of gene therapy DNA vector production based on gene therapy DNA vector VTvafl7 carrying the NOS2, NOS3, VIP, KCNMA1, CGRP therapeutic gene as per claim 1, 2, 3, 4, or 5 that involves obtaining each of gene therapy DNA vectors: VTvafl7-NOS2, or VTvafl7-NOS3, or VTvafl7-VIP, or VTvafl7-KCNMA1, or VTvafl7-CGRP as follows: the coding region of the NOS2, NOS3, VIP, KCNMA1, CGRP therapeutic gene is cloned to gene therapy DNA vector VTvafl7, and gene therapy DNA vector VTvafl7-NOS2, SEQ ID No. 1, or VTvafl7-NOS3, SEQ ID No. 2, or VTvafl7-VIP, SEQ ID No. 3, or VTvafl7-KCNMA1, SEQ ID No. 4 or VTvafl7-CGRP, SEQ ID No. 5, respectively, is obtained, while the coding region of the NOS2, or NOS3, or VIP, or KCNMA1, or CGRP therapeutic gene is obtained by isolating total RNA from the human biological tissue sample followed by the reverse transcription reaction and PCR amplification using the obtained oligonucleotides and cleaving the amplification product by corresponding restriction endonucleases, while cloning to the gene therapy DNA vector VTvafl7 is performed by Sa1I and KpnI, or BamHI and EcoRI restriction sites, while the selection is performed without antibiotics.
at the same time, the following oligonucleotides produced for this purpose are used during gene therapy DNA vector VTvafl7-NOS2, SEQ ID No. 1 production for the reverse transcription reaction and PCR amplification:
NOS2_F
ATCGTCGACCACCATGGCCTGTCCTTGGAAATTTC,
NOS2_R
CGGTACCTCAGAGCGCTGACATCTCCAGG,
and the cleaving of amplification product and cloning of the coding region of NOS2 gene to gene therapy DNA vector VTvafl7 is performed by SalI and KpnI restriction endonucleases,
at the same time, the following oligonucleotides produced for this purpose are used during gene therapy DNA vector VTvafl7-NOS3, SEQ ID No. 2 production for the reverse transcription reaction and PCR amplification:
NOS3_F
GACAAGCTTCCACCATGGGCAACTTGAAGAG,
NOS3_R
GGAATTCAGGGGCTGTTGGTGTCTGAGCCG,
and the cleaving of amplification product and cloning of the coding region of NOS3 gene to gene therapy DNA vector VTvafl7 is performed by HindIIII H EcoRI restriction endonucleases,
at the same time, the following oligonucleotides produced for this purpose are used during gene therapy DNA vector VTvafl7-VIP, SEQ ID No. 3 production for the reverse transcription reaction and PCR amplification:
VIP_F
AGGATCCACCATGGACACCAGAAATAAGGCCCAG,
VIP_R
GGAATTCATTTTTCTAACTCTTCTGGAAAG,
and the cleaving of amplification product and cloning of the coding region of VIP gene to gene therapy DNA vector VTvafl7 is performed by BamHI H EcoRI restriction endonucleases,
at the same time, the following oligonucleotides produced for this purpose are used during gene therapy DNA vector VTvafl7-KCNMA1, SEQ ID No. 4 production for the reverse transcription reaction and PCR amplification:
KCNMA1_F
AGGATCCGGTACCGAGGAGATCTGCCGCCGCGATCGCCATG,
KCNMA1_R
ACCAAGCTTATCTGTAAACCATTTCTTTTCTG,
and the cleaving of amplification product and cloning of the coding region of KCNMA1 gene to gene therapy DNA vector VTvafl7 is performed by BamHI H EcoRI restriction endonucleases,
at the same time, the following oligonucleotides produced for this purpose are used during gene therapy DNA vector VTvafl7-CGRP, SEQ ID No. 5 production for the reverse transcription reaction and PCR amplification:
CGRP_F
AGGATCCGGACGTCATGGAAGTGAAGGATGCCAATT,
CGRP_R
GGAATTCCTATGCTGGGTCCTCTTCGTCCATTG,
and the cleaving of amplification product and cloning of the coding region of CGRP gene to gene therapy DNA vector VTvafl7 is performed by BamHI H EcoRI restriction endonucleases.

9. A method of use of the gene therapy DNA vector based on gene therapy DNA vector VTvafl7 carrying NOS2, NOS3, VIP, KCNMA1, and CGRP therapeutic gene as per claim 1, 2, 3, 4, or 5 for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction that involves transfection of the cells of patient or animal organs and tissues with the selected gene therapy DNA vector carrying the therapeutic gene based on gene therapy DNA vector VTvafl7, or several selected gene therapy DNA vectors carrying therapeutic genes based on gene therapy DNA vector VTvafl7, from the group of constructed gene therapy DNA vectors carrying therapeutic genes based on gene therapy DNA vector VTvafl7 and/or injection of autologous cells of said patient or animal transfected by the selected gene therapy DNA vector carrying therapeutic gene based on gene therapy DNA vector VTvafl7 or several selected gene therapy DNA vectors carrying the therapeutic genes based on gene therapy DNA vector VTvafl7 from the constructed gene therapy DNA vectors carrying therapeutic genes based on gene therapy DNA vector VTvafl7 into the organs and tissues of the same patient or animal and/or the injection of the selected gene therapy DNA vector carrying therapeutic gene based on gene therapy DNA vector VTvafl7 or several selected gene therapy DNA vectors carrying therapeutic genes based on gene therapy DNA vector VTvafl7 from the group of constructed gene therapy DNA vectors carrying therapeutic genes based on gene therapy DNA vector VTvafl7 into the organs and tissues of the same patient or animal, or the combination of the indicated methods

10. A method of production of strain for construction of a gene therapy DNA vector as per claim 1, 2, 3, 4 or 5 for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction that involves making electrocompetent cells of Escherichia coli strain SCS110-AF and subjecting these cells to electroporation with gene therapy DNA vector VTvafl7-NOS2, or gene therapy DNA vector VTvafl7-NOS3, or gene therapy DNA vector VTvafl7-VIP, or gene therapy DNA vector VTvafl7-KCNMA1, or gene therapy DNA vector VTvafl7-CGRP. After that, the cells are poured into agar plates (Petri dishes) with a selective medium containing yeastrel, peptone, 6% sucrose, and 10 μg/ml of chloramphenicol, and as a result, Escherichia coli strain SCS110-AF/VTvafl7-NOS2 or Escherichia coli strain SCS110-AF/VTvafl7-NOS3, or Escherichia coli strain SCS110-AF/VTvafl7-VIP, or Escherichia coli strain SCS110-AF/VTvafl7-KCNMA1, or Escherichia coli strain SCS110-AF/VTvafl7-CGRP is obtained.

11. Escherichia coli strain SCS110-AF/VTvafl7-NOS2, obtained as per claim 10 carrying the gene therapy DNA vector VTvafl7-NOS2 for production thereof allowing for antibiotic-free selection during the production of the gene therapy DNA vector for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction.

12. Escherichia coli strain SCS110-AF/VTvafl7-NOS3, obtained as per claim 10 carrying the gene therapy DNA vector VTvafl7-NOS3 for production thereof allowing for antibiotic-free selection during the production of the gene therapy DNA vector for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction.

13. Escherichia coli strain SCS110-AF/VTvafl7-VIP, obtained as per claim 10 carrying the gene therapy DNA vector VTvafl7-VIP for production thereof allowing for antibiotic-free selection during the production of the gene therapy DNA vector for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction.

14. Escherichia coli strain SCS110-AF/VTvafl7-KCNMA1, obtained as per claim 10 carrying the gene therapy DNA vector VTvafl7-KCNMA1 for production thereof allowing for antibiotic-free selection during the production of the gene therapy DNA vector for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction.

15. Escherichia coli strain SCS110-AF/VTvafl7-CGRP, obtained as per claim 10 carrying the gene therapy DNA vector VTvafl7-CGRP for production thereof allowing for antibiotic-free selection during the production of the gene therapy DNA vector for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction.

16. A method of production on an industrial scale of gene therapy DNA vector based on gene therapy DNA vector VTvafl7 carrying the NOS2, or NOS3, or VIP, or KCNMA1, or CGRP therapeutic gene as per claim 1, 2, 3, 4 or 5 for treatment of diseases associated with disorders of neurotransmission, antimicrobial and antitumor immunity, vasomotion of various organs and tissues, for stimulation of myocardial contractile function, vasodilation, increase of glycogenolysis, decrease of arterial blood pressure, relaxation of smooth muscle, and treatment of erectile dysfunction that involves production of gene therapy DNA vector VTvafl7-NOS2, or gene therapy DNA vector VTvafl7-NOS3, or gene therapy DNA vector VTvafl7-VIP, or gene therapy DNA vector VTvafl7-KCNMA1, or gene therapy DNA vector VTvafl7-CGRP by inoculating a culture flask containing the prepared medium with seed culture selected from Escherichia coli strain CS110-AF/VTvafl7-NOS2, or Escherichia coli strain SCS110-AF/VTvafl7-NOS3, or Escherichia coli strain SCS110-AF/VTvafl7-VIP, or Escherichia coli strain SCS110-AF/VTvafl7-KCNMA1, or Escherichia coli strain SCS110-AF/VTvafl7-CGRP then the cell culture is incubated in an incubator shaker and transferred to an industrial fermenter, then grown to a stationary phase, then the fraction containing the target DNA product is extracted, multi-stage filtered, and purified by chromatographic methods.


SEQUENCE LISTING (25 sequences)

SEQ ID NO: 1 - DNA, 6625 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatggcctgtccttggaaatttctgttcaagaccaaattccaccagtatgcaatgaatggggaaaaagacatcaacaacaatgtggagaaagccccctgtgccacctccagtccagtgacacaggatgaccttcagtatcacaacctcagcaagcagcagaatgagtccccgcagcccctcgtggagacgggaaagaagtctccagaatctctggtcaagctggatgcaaccccattgtcctccccacggcatgtgaggatcaaaaactggggcagcgggatgactttccaagacacacttcaccataaggccaaagggattttaacttgcaggtccaaatcttgcctggggtccattatgactcccaaaagtttgaccagaggacccagggacaagcctacccctccagatgagcttctacctcaagctatcgaatttgtcaaccaatattacggctccttcaaagaggcaaaaatagaggaacatctggccagggtggaagcggtaacaaaggagatagaaacaacaggaacctaccaactgacgggagatgagctcatcttcgccaccaagcaggcctggcgcaatgccccacgctgcattgggaggatccagtggtccaacctgcaggtcttcgatgcccgcagctgttccactgcccgggaaatgtttgaacacatctgcagacacgtgcgttactccaccaacaatggcaacatcaggtcggccatcaccgtgttcccccagcggagtgatggcaagcacgacttccgggtgtggaatgctcagctcatccgctatgctggctaccagatgccagatggcagcatcagaggggaccctgccaacgtggaattcactcagctgtgcatcgacctgggctggaagcccaagtacggccgcttcgatgtggtccccctggtcctgcaggccaatggccgtgaccctgagctcttcgaaatcccacctgaccttgtgcttgaggtggccatggaacatcccaaatacgagtggtttcgggaactggagctaaagtggtacgccctgcctgcagtggccaacatgctgcttgaggtgggcggcctggagttcccagggtgccccttcaatggctggtacatgggcacagagatcggagtccgggacttctgtgacgtccagcgctacaacatcctggaggaagtgggcaggagaatgggcctggaaacgcacaagctggcctcgctctggaaagaccaggctgtcgttgagatcaacattgctgtgctccatagtttccagaagcagaatgtgaccatcatggaccaccactcggctgcagaatccttcatgaagtacatgcagaatgaataccggtcccgtgggggctgcccggcagactggatttggctggtccctcccatgtctgggagcatcacccccgtgtttcaccaggagatgctgaactacgtcctgtcccctttctactactatcaggtagaggcctggaaaacccatgtctggcaggacgagaagcggagacccaagagaagagagattccattgaaagtcttggtcaaagctgtgctctttgcctgtatgctgatgcgcaagacaatggcgtcccgagtcagagtcaccatcctctttgcgacagagacaggaaaatcagaggcgctggcctgggacctgggggccttattcagctgtgccttcaaccccaaggttgtctgcatggataagtacaggctgagctgcctggaggaggaacggctgctgttggtggtgaccagtacgtttggcaatggagactgccctggcaatggagagaaactgaagaaatcgctcttcatgctgaaagagctcaacaacaaattcaggtacgctgtgtttggcctcggctccagcatgtaccctcggttctgcgcctttgctcatgacattgatcagaagctgtcccacctgggggcctctcagctcaccccgatgggagaaggggatgagctcagtgggcaggaggacgccttccgcagctgggccgtgcaaaccttcaaggcagcctgtgagacgtttgatgtccgaggcaaacagcacattcagatccccaagctctacacctccaatgtgacctgggacccgcaccactacaggctcgtgcaggactcacagcctttggacctcagcaaagccctcagcagcatgcatgccaagaacgtgttcaccatgaggctcaaatctcggcagaatctacaaagtccgacatccagccgtgccaccatcctggtggaactctcctgtgaggatggccaaggcctgaactacctgccgggggagcaccttggggtttgcccaggcaaccagccggccctggtccaaggtatcctggagcgagtggtggatggccccacaccccaccagacagtgcgcctggaggccctggatgagagtggcagctactgggtcagtgacaagaggctgcccccctgctcactcagccaggccctcacctacttcctggacatcaccacacccccaacccagctgctgctccaaaagctggcccaggtggccacagaagagcctgagagacagaggctggaggccctgtgccagccctcagagtacagcaagtggaagttcaccaacagccccacattcctggaggtgctagaggagttcccgtccctgcgggtgtctgctggcttcctgctttcccagctccccattctgaagcccaggttctactccatcagctcctcccgggatcacacgcccacagagatccacctgactgtggccgtggtcacctaccacacccgagatggccagggtcccctgcaccacggcgtctgcagcacatggctcaacagcctgaagccccaagacccagtgccctgctttgtgcggaatgccagcggcttccacctccccgaggatccctcccatccttgcatcctcatcgggcctggcacaggcatcgcgcccttccgcagtttctggcagcaacggctccatgactcccagcacaagggagtgcggggaggccgcatgaccttggtgtttgggtgccgccgcccagatgaggaccacatctaccaggaggagatgctggagatggcccagaagggggtgctgcatgcggtgcacacagcctattcccgcctgcctggcaagcccaaggtctatgttcaggacatcctgcggcagcagctggccagcgaggtgctccgtgtgctccacaaggagccaggccacctctatgtttgcggggatgtgcgcatggcccgggacgtggcccacaccctgaagcagctggtggctgccaagctgaaattgaatgaggagcaggtcgaggactatttctttcagctcaagagccagaagcgctatcacgaagatatctttggtgctgtatttccttacgaggcgaagaaggacagggtggcggtgcagcccagcagcctggagatgtcagcgctctgaggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 2 - DNA, 6774 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgacaagcttccaccatgggcaacttgaagagcgtggcccaggagcctgggccaccctgcggcctggggctggggctgggccttgggctgtgcggcaagcagggcccagccaccccggcccctgagcccagccgggccccagcatccctactcccaccagcgccagaacacagccccccgagctccccgctaacccagcccccagaggggcccaagttccctcgtgtgaagaactgggaggtggggagcatcacctatgacaccctcagcgcccaggcgcagcaggatgggccctgcaccccaagacgctgcctgggctccctggtatttccacggaaactacagggccggccctcccccggccccccggcccctgagcagctgctgagtcaggcccgggacttcatcaaccagtactacagctccattaagaggagcggctcccaggcccacgaacagcggcttcaagaggtggaagccgaggtggcagccacaggcacctaccagcttagggagagcgagctggtgttcggggctaagcaggcctggcgcaacgctccccgctgcgtgggccggatccagtgggggaagctgcaggtgttcgatgcccgggactgcaggtctgcacaggaaatgttcacctacatctgcaaccacatcaagtatgccaccaaccggggcaaccttcgctcggccatcacagtgttcccgcagcgctgccctggccgaggagacttccgaatctggaacagccagctggtgcgctacgcgggctaccggcagcaggacggctctgtgcggggggacccagccaacgtggagatcaccgagctctgcattcagcacggctggaccccaggaaacggtcgcttcgacgtgctgcccctgctgctgcaggccccagatgatcccccagaactcttccttctgccccccgagctggtccttgaggtgcccctggagcaccccacgctggagtggtttgcagccctgggcctgcgctggtacgccctcccggcagtgtccaacatgctgctggaaattgggggcctggagttccccgcagcccccttcagtggctggtacatgagcactgagatcggcacgaggaacctgtgtgaccctcaccgctacaacatcctggaggatgtggctgtctgcatggacctggatacccggaccacctcgtccctgtggaaagacaaggcagcagtggaaatcaacgtggccgtgctgcacagttaccagctagccaaagtcaccatcgtggaccaccacgccgccacggcctctttcatgaagcacctggagaatgagcagaaggccagggggggctgccctgcagactgggcctggatcgtgccccccatctcgggcagcctcactcctgttttccatcaggagatggtcaactatttcctgtccccggccttccgctaccagccagacccctggaaggggagtgccgccaagggcaccggcatcaccaggaagaagacctttaaagaagtggccaacgccgtgaagatctccgcctcgctcatgggcacggtgatggcgaagcgagtgaaggcgacaatcctgtatggctccgagaccggccgggcccagagctacgcacagcagctggggagactcttccggaaggcttttgatccccgggtcctgtgtatggatgagtatgacgtggtgtccctcgaacacgagacgctggtgctggtggtaaccagcacatttgggaatggggatcccccggagaatggagagagctttgcagctgccctgatggagatgtccggcccctacaacagctcccctcggccggaacagcacaagagttataagatccgcttcaacagcatctcctgctcagacccactggtgtcctcttggcggcggaagaggaaggagtccagtaacacagacagtgcaggggccctgggcaccctcaggttctgtgtgttcgggctcggctcccgggcatacccccacttctgcgcctttgctcgtgcggtggacacacggctggaggaactgggcggggagcggctgctgcagctgggccagggcgacgagctgtgcggccaggaggaggccttccgaggctgggcccaggctgccttccaggccgcctgtgagaccttctgtgtgggagaggatgccaaggccgccgcccgagacatcttcagccccaaacggagctggaagcgccagaggtaccggctgagcgcccaggccgagggcctgcagttgctgccaggtctgatccacgtgcacaggcggaagatgttccaggctacaatccgctcagtggaaaacctgcaaagcagcaagtccacgagggccaccatcctggtgcgcctggacaccggaggccaggaggggctgcagtaccagccgggggaccacataggtgtctgcccgcccaaccggcccggccttgtggaggcgctgctgagccgcgtggaggacccgccggcgcccactgagcccgtggcagtagagcagctggagaagggcagccctggtggccctccccccggctgggtgcgggacccccggctgcccccgtgcacgctgcgccaggctctcaccttcttcctggacatcacctccccacccagccctcagctcttgcggctgctcagcaccttggcagaagagcccagggaacagcaggagctggaggccctcagccaggatccccgacgctacgaggagtggaagtggttccgctgccccacgctgctggaggtgctggagcagttcccgtcggtggcgctgcctgccccactgctcctcacccagctgcctctgctccagccccggtactactcagtcagctcggcacccagcacccacccaggagagatccacctcactgtagctgtgctggcatacaggactcaggatgggctgggccccctgcactatggagtctgctccacgtggctaagccagctcaagcccggagaccctgtgccctgcttcatccggggggctccctccttccggctgccacccgatcccagcttgccctgcatcctggtgggtccaggcactggcattgcccccttccggggattctggcaggagcggctgcatgacattgagagcaaagggctgcagcccactcccatgactttggtgttcggctgccgatgctcccaacttgaccatctctaccgcgacgaggtgcagaacgcccagcagcgcggggtgtttggccgagtcctcaccgccttctcccgggaacctgacaaccccaagacctacgtgcaggacatcctgaggacggagctggctgcggaggtgcaccgcgtgctgtgcctcgagcggggccacatgtttgtctgcggcgatgttaccatggcaaccaacgtcctgcagaccgtgcagcgcatcctggcgacggagggcgacatggagctggacgaggccggcgacgtcatcggcgtgctgcgggatcagcaacgctaccacgaagacattttcgggctcacgctgcgcacccaggaggtgacaagccgcatacgcacccagagcttttccttgcaggagcgtcagttgcggggcgcagtgccctgggcgttcgaccctcccggctcagacaccaacagcccctgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 3 - DNA, 3652 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatggacaccagaaataaggcccagctccttgtgctcctgactcttctcagtgtgctcttctcacagacttcggcatggcctctttacagggcaccttctgctctcaggttgggtgacagaataccctttgagggagcaaatgaacctgatcaagtttcattaaaagaagacattgacatgttgcaaaatgcattagctgaaaatgacacaccctattatgatgtatccagaaatgccaggcatgctgatggagttttcaccagtgacttcagtaaactcttgggtcaactttctgccaaaaagtaccttgagtctcttatgggaaaacgtgttagtaacatctcagaagaccctgtaccagtcaaacgtcactcagatgcagtcttcactgacaactatacccgccttagaaaacaaatggctgtaaagaaatatttgaactcaattctgaatggaaagaggagcagtgagggagaatctcccgactttccagaagagttagaaaaatgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 4 - DNA, 6731 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccggtaccgaggagatctgccgccgcgatcgccatggcaaatggtggcggcggcggcggcggcagcagcggcggcggcggcggcggcggaggcagcagtcttagaatgagtagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctactttggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagccgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgaagccgaaagcgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactggtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaacagaaaagaaatggtttacagataagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 5 - DNA, 3586 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccggacgtcatggaagtgaaggatgccaattctgcgcttctcagtaactacgaggtatttcagttactaactgatctgaaagagcagcgtaaagaaagtggaaagaataaacacagctctgggcaacagaacttgaacactatcacctatgaaacgttaaaatacatatcaaaaacaccatgcaggcaccagagtcctgaaattgtcagagaatttctcacagcattgaaaagccacaagttgaccaaagctgagaagctccagctgctgaaccaccggcctgtgactgctgtggagatccagctgatggtggaagagagtgaagagcggctcacggaggagcagattgaagctcttctccacaccgtcaccagcattctgcctgcagagccagaggctgagcagaagaagaatacaaacagcaatgtggcaatggacgaagaggacccagcataggaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttg

SEQ ID NO: 6 - DNA, 35 bp, Artificial Sequence
  Oligonucleotide NOS2_F
atcgtcgaccaccatggcctgtccttggaaatttc

SEQ ID NO: 7 - DNA, 29 bp, Artificial Sequence
  Oligonucleotide NOS2_R
cggtacctcagagcgctgacatctccagg

SEQ ID NO: 8 - DNA, 20 bp, Artificial Sequence
  Oligonucleotide NOS2_SF
aacgtgttcaccatgaggct

SEQ ID NO: 9 - DNA, 20 bp, Artificial Sequence
  Oligonucleotide NOS2_SR
ctctcaggctcttctgtggc

SEQ ID NO: 10 - DNA, 31 bp, Artificial Sequence
  Oligonucleotide NOS3_F
gacaagcttccaccatgggcaacttgaagag

SEQ ID NO: 11 - DNA, 30 bp, Artificial Sequence
  Oligonucleotide NOS3_R
ggaattcaggggctgttggtgtctgagccg

SEQ ID NO: 12 - DNA, 20 bp, Artificial Sequence
  Oligonucleotide NOS3_SF
gacccactggtgtcctcttg

SEQ ID NO: 13 - DNA, 20 bp, Artificial Sequence
  Oligonucleotide NOS3_SR
ctccgtttggggctgaagat

SEQ ID NO: 14 - DNA, 34 bp, Artificial Sequence
  Oligonucleotide VIP_F
aggatccaccatggacaccagaaataaggcccag

SEQ ID NO: 15 - DNA, 30 bp, Artificial Sequence
  Oligonucleotide VIP_R
ggaattcatttttctaactcttctggaaag

SEQ ID NO: 16 - DNA, 20 bp, Artificial Sequence
  Oligonucleotide VIP_SF
ccttctgctctcaggttggg

SEQ ID NO: 17 - DNA, 20 bp, Artificial Sequence
  Oligonucleotide VIP_SR
ccctcactgctcctctttcc

SEQ ID NO: 18 - DNA, 41 bp, Artificial Sequence
  Oligonucleotide KCNMA1_F
aggatccggtaccgaggagatctgccgccgcgatcgccatg

SEQ ID NO: 19 - DNA, 32 bp, Artificial Sequence
  Oligonucleotide KCNMA1_R
accaagcttatctgtaaaccatttcttttctg

SEQ ID NO: 20 - DNA, 20 bp, Artificial Sequence
  Oligonucleotide KCNMA1_SF
gagagagccgaagccgaaag

SEQ ID NO: 21 - DNA, 20 bp, Artificial Sequence
  Oligonucleotide KCNMA1_SR
acccttgggaattagcctgc

SEQ ID NO: 22 - DNA, 36 bp, Artificial Sequence
  Oligonucleotide CGRP_F
aggatccggacgtcatggaagtgaaggatgccaatt

SEQ ID NO: 23 - DNA, 33 bp, Artificial Sequence
  Oligonucleotide CGRP_R
ggaattcctatgctgggtcctcttcgtccattg

SEQ ID NO: 24 - DNA, 21 bp, Artificial Sequence
  Oligonucleotide CGRP_SF
actgatctgaaagagcagcgt

SEQ ID NO: 25 - DNA, 20 bp, Artificial Sequence
  Oligonucleotide CGRP_SR
agaatgctggtgacggtgtg